(19) United States
(12) Patent Application Publication
(10) Pub. No.: US 2002/0055536 A1
(43) Pub. Date: May 9, 2002

(54) System and method for structure-based drug design that includes accurate prediction of binding free energy

(76) Inventors: Robert S. DeWitte, Gurnee, IL (US); Eugene I. Shakhnovich, Lexington, MA (US)

Correspondence Address: HALE AND DORR, LLP, 60 STATE STREET, BOSTON, MA, 02109

(21) Appl. No.: 09/897,584

(22) Filed: Jun. 29, 2001

Related U.S. Application Data
(63) Continuation of application No. 09/220,363, filed on Dec. 24, 1998, now abandoned.
Application No. 09/220,363 is a continuation-in-part of application No. 08/741,866, filed on Sep. 26, 1996, now Pat. No. 5,854,992.

(51) Int. Cl. A61K 31/335; G06F 19/00; G01N 33/48; G01N 33/50; A61K 31/70

(52) U.S. Cl. 514/449; 514/23; 702/19

(57) ABSTRACT

A system and method for providing improved de novo structure-based drug design that includes a method for more accurately predicting binding free energy. The system and method use a coarse-graining model with corresponding knowledge based potential data to grow ligand candidates and libraries of ligand candidates. In light of the present inventions using the coarse-graining model, the novel growth method of the present invention uses a metropolis Monte Carlo selection process which result in a low energy structure that is not necessarily the lowest energy structure, yet results in a better ligand candidate.

This application is a continuation-in-part of copending U.S. Ser. No. 08/741,866, filed Sep. 26, 1996, which is hereby incorporated by reference.

### FIELD OF THE INVENTION

The present invention generally relates to systems and methods for de novo structure-based drug design. More specifically, the present invention relates to systems and methods for de novo structure-based drug design that includes a method for accurately predicting the binding free energy in building novel therapeutic molecules or ligands.

### BACKGROUND OF THE INVENTION

Finding leads and optimizing the leads that are found are the goals in the development of molecules or ligands that are useful in combating disease and other abnormal body conditions. In recent years, this has involved using, among other things, a molecular similarity method of drug design. This is just one of a number of methods for determining useful leads in the search for new or improved bioactive, therapeutic molecules or ligands. The molecular similarity method of drug design is based on evaluating a large range of chemical structures to find those that show either similarity with each other or complementarity with a biochemical target structure. Chemical structures identified in this manner, usually have a reasonably high probability of binding at the target structure. This search for appropriate chemical structures is enhanced and improved by computer technology which uses search algorithms to search large databases of chemical structures.

Other methods to develop desired leads or ligands has been to use high throughput screening or combinatorial chemistry. These conventional methods have been proven, in certain circumstances, to derive desired leads and ligands.

Structure-based molecular design is yet another method to identify lead molecules for drug design. This method is based on the premise that good inhibitors possess significant structural and chemical complementarity with their target receptors. This design method can create molecules with specific properties that make them conducive for binding to the target site. The molecular structures that are designed by the structure-based design process are meant to interact with biochemical targets, for example, whose three-dimensional structures are known.

The structure-based drug design method has distinct advantages over prior art methods. One important advantage is that the structure-based drug design method provides over traditional methods of lead discovery and optimization is an awareness of the intermolecular interactions that are possible.

One goal of the structure-based drug design method is to identify lead molecules. This may be accomplished in a variety of ways. However, the principal generic steps in most structure-based drug design methods are: (1) to identify and determine the structure of the receptor site; (2) to use theoretical principles and experimental data to propose a series of putative ligands that will bind to the receptor sites (which ligands are synthesized and tested for their complementarity); (3) to make a determination of the structure of the receptor/ligand complexes that are successful in binding at low free energy levels; and (4) to iterate steps 2 and 3 in an effort to further enhance binding.

The success of a structure-based drug design method, as can be surmised, may be enhanced through the use of advanced methods of computation which will expedite the identification of key molecular fragments (which may then joined to form molecules) or whole molecules (either from a database of existing compounds or through a molecular growth algorithm). These computational advances enhance the ability to develop molecules or ligands which will successfully bind to macromolecular receptor sites.

One such advance, computational docking of molecules, takes into account the structure of a receptor site and an estimation of the binding affinity of the potential drug design candidate for the macromolecular receptor. This serves as a basis for structure-based drug design through three-dimensional databases of synthesized molecules. There are considerable mathematical challenges that must be addressed and conquered in tackling docking. These include the absence of a clearly best method to mathematically describe the molecular shape of the receptor site and ligand, the packing of the irregular objects together (the receptor and ligand surfaces which are to be joined), and the search issues that are related to graph theory, namely, the study of isomorphous subgraphs to make the matches of a receptor site and ligand. Docking of ligands has three components: (1) site/ligand description, (2) juxtaposition of the ligand and the site frames of reference, and (3) the evaluation of the complementarity. With regard to the site/ligand description, the atomic coordinates of the receptor macromolecules are obtained through methods, such as X-ray crystallography, nuclear magnetic resonance (NMR), or homology modeling. Site descriptions simply may be the atomic coordinates of the receptor site; however, some notion of the chemical properties of the atoms is needed if there are hopes to measure chemical complementarity, and not just spatial complementarity. Site volume also may be defined if a site boundary is not identifiable. Ligand descriptions closely parallel site descriptions.

Structures for a large number of organic and inorganic compounds have been determined experimentally using X-ray and neutron crystallography technology. If such structures are not so defined, the approximate three-dimensional coordinates for the associated compounds may be obtained by using commercially available computer software products that use a combination of force fields and heuristic rules to generate atomic coordinates.

In considering the juxtaposition of the ligands and receptor sites, the general desire in molecular docking is to obtain the lowest free energy structure for the receptor-ligand complex. Moreover, this search for the lowest free energy selects the best fit at each position of the structure with regard to the lowest binding free energy and selection criteria. To attempt to achieve this result (1) databases of putative ligands are searched and identified ligands are ranked according to their respective interactive energies with a particular receptor site and (2) computational studies are made of the geometry of particular complexes.

In evaluating the complementarity of a receptor site and ligand, of interest for ligand design is the free energy of binding (_g_{binding}). This may be calculated directly using free energy perturbation methods, if an accurate geometric model of the ligand-receptor complex is available. Free energy calculations generally require a great deal of computational time, i.e., in the order of days. In light of this, there have been many proposed simplifications for calculating free energy; however, these simplifications have tended to add to the inaccuracy of the free energy determination. This degradation of the accuracy is unacceptable, and, as such, is there is a demand for a method to compute free energy in what is considered a reasonable amount of time, which is in the minute or less range, and accuracy does not suffer.

The calculation or prediction of free energy can be effected by any of a number of commercially available software programs. A few of these programs and their computational strategies will be discussed in greater detail subsequently.

It is known to use one of two methods to automatically search databases that contain large amounts of data relating to fragments that can be used for building molecules or ligands for developing lead candidates. A first method is the Geometric method that matches ligand and receptor site descriptors. A second method is to align the ligand and receptor by minimizing the ligand and receptor interactive energy. To fulfill the requirements of this method, energy-driven searching may be based on molecular dynamics (“MD”) and traditional Monte Carlo (“MC”) simulations. These methods, however, require a tremendous amount of computational time. Finding the lowest energy state of a given ligand-receptor complex using either of these methods is a fundamental problem.

Attempts to find ways around the actual calculation of free-binding energy has resulted in search methods based on descriptors, grids, and fragments. Looking to descriptor matching methods, an analysis is made of the proposed receptor region at which binding is to take place. Ligand atoms are then positioned at the best locations at the site. This gives an approximated ligand-receptor configuration that may then be refined by optimization. Descriptor matching methods are reasonably fast and provide a good sampling of the region of interest at the receptor site. Many of the descriptor matching methods use algorithms that employ combinational search strategies. As such, small changes in parameter values can cause the computational time required to become unreasonably long.

DOCK is one of the earliest descriptor matching programs. DOCK software was developed at the University of California at San Francisco and provides a method that attempts to solve the problem by developing a drug by creating a negative image of the target site, searching a database for a similar ligand, placing putative ligands into the site, and evaluating the quality of the fit.

In using the DOCK software, an important factor is the accurate prediction of binding free energy. In favorable cases, the accuracy can reach as high as 1-2 kcal/mol. In operation, DOCK uses spheres locally complementary to the receptor surface to create a space-filling negative image of the receptor site. Several ligand atoms are matched with receptor spheres to generate chiral orientations of the ligand in the site. Databases of small molecules are searched for candidates that complement the structure of the receptor site.

There are problems with DOCK which result in predictions that are, in fact, not as accurate as desired. Further, DOCK is unable to suggest any novel structures, it can only search for what is in a database.

CAVEAT software, also a descriptor matching method, is based on directional characterization of ligands. CAVEAT was developed at the University of California at Berkeley. This program searches for ligands with atoms located along specified vectors. The vectors are derived from structural information from known complexes. CAVEAT focuses on searching ligand databases to find templates as starting points for chemical structures.

FOUNDATION software provides a descriptor matching method that attempts to combine models of crucial ligand atoms and structure-based models. In using FOUNDATION, the searcher identifies atom and binding types that the candidate molecule must possess. FOUNDATION relies heavily on the detailed atom-type, bond-type, chain length, and topology constraints provided by the searcher to restrict the search. FOUNDATION only considers the steric component of the active site and relies on the matching information to find chemically complementary ligands. The tight constraints that are required by the FOUNDATION program restrict the candidates to one orientation at the receptor site.

CLIX software, developed at CSIRO, an Australian company, resembles DOCK by using the receptor site to define possible binding configurations. CLIX relies on an elaborate chemical description of the receptor site. This program uses fewer receptor-ligand matches than does DOCK. CLIX also evaluates interaction energies at the receptor sites.

Grid search methods are used to sample the six degrees of freedom of the orientation space. These methods identity an approximate solution, which cannot be guaranteed with discrete sampling methods. Accuracy is limited by the step size used in the search of the various positions. The size of step also determines the time of the search, i.e., the greater the number of incremental steps, the greater the search time. Methods that use additional sampling in regions of high complementarity can overcome this problem.

A first type of grid search method is a side chain spheres method. This method explores protein-protein complexes using simplified sphere representations of side chain atoms and a grid search of four rigid degrees of freedom. This program uses surface evaluation algorithms, full molecular force-field evaluations of complexes, and simulated annealing to refine initial docking structures.

A second type of grid search method is a soft docking method. According to this method, receptor and ligand surfaces are divided into cubes to generate the translational part of the search. A pure rotational grid search is conducted on the sample ligand at orientations in discreet angular increments. The accuracy is limited by size. Run-time scaling is the cube of the rotational step size and is a product of the number of the receptor-ligand surface points.

Fragment-joining methods identify regions of high complementarity by docking functional groups independently into receptors. These methods are not particularly bothered by rigid ligand issues because of the added combinational search. Fragment-joining methods suggest unsynthesized compounds, but connecting the fragments in sensible, synthetically accessible patterns is difficult. Fragment-joining methods have the problem that there is a need to connect functional groups to form complete molecules while maintaining the fragments at the geometric positions of lowest energy.

GROW is a fragment-joining method which has been used to design peptides complementary to proteins of a known structure. The software was developed at Upjohn Laboratories, Kalamazoo, Mich. In operation, a seed amino acid is placed in the receptor site followed by iterative additions of amino acids. Conformations are chosen from a library of precalculated low-energy forms. At each addition of a peptide, the peptide-receptor complex is minimized and evaluated. Only the best 10-100 low energy structures are kept at any stage.

GROWMOL software generates molecules by evaluating each new atom added to molecules according to the chemical complementarity of the atom to nearby atoms on the molecule. A Boltzmann weighing factor is used to bias the probability of selection towards atoms with a high complementarity score. The chemical complementarity is determined by calculating the number of hydrophobic contacts (i.e. the number of ligand carbons other than carbonyl carbons which occupy a pre-defined “hydrophobic zone”) and the number of hydrogen bond (i.e. the number of ligand hydrogens in a pre-defined “hydrogen acceptor zone” plus the number of ligand oxygens found in a pre-defined “hydrogen bond donor zone”).

GROWBUILD software grows molecules by the addition of fragments from a library consisting of a single functional group such as a hydroxy, a carbonyl, or a benzene ring. At each setup, possible fragment additions are evaluated according to the molecular mechanics energy and one of the best is randomly chosen. No information about critical binding regions is used in the beginning to identify disconnected regions of the active site which must be filled.

HOOK, developed at Harvard University, Cambridge, Mass., is a fragment-joining method that finds hot spots in receptor sites by looking for low energy locations for functional groups. HOOK uses random placement of many copies of several functional fragments followed by molecular dynamics.

Multiple Start Monte Carlo methods also have been used as fragment-joining methods. These methods conduct searches of databases for fragments of a ligand to dock at the receptor site.

MCSS-HOOK-DLD methods involve the location of favorable interaction sites for molecular fragments by performing a multiple copy simultaneous search (MCSS). In such a search, the protein is subject to the average potential field of the ligands using the CHARMM empirical force field. The resulting interaction sites, unlike with GRID, contain orientation information and can be linked together with bonding force fields and linker sp³ and sp₂ carbon atoms via DLD (dynamic ligand design) or molecular fragments in a database (HOOK). The unfortunate aspect of MCSS-based approach is the large amount of computation required, several days preparation time on a modern workstation followed by approximately an hour of computation for each ligand candidate.

BUILDER software, uses a family of docked structures to provide an irregular lattice of controllable density. This lattice can be searched for paths that link molecular fragments.

LUDI is a fragment-joining method that proposes inhibitors by connecting fragments that dock into microsites on the receptor. LUDI was developed at BASF, Stuttgard, Germany. The fragments are from a predetermined list of molecular fragments. The microsites are defined by hydrogen bonding and hydrophobic groups. Ligand pseudoatom positions are generated within microsites on the basis of an appropriate angle and distance minima for various interactions. The fragments identified are connected using linear chains composed of one or more of 12 functional groups.

GRID software is a hybrid grid/fragment-joining method that places small fragment probes at many regularly spaced grid points within an active receptor site. This program, developed at Oxford University, England, has been found to reproduce the positions of important hydrogen bonding groups. GRID uses empirical hydrogen bonding interaction potential and spherical representations of functional groups to generate affinity contours for various molecular fragments. This identifies regions of high and low affinity. The contours may be used to guide chemical intuition or as an input for other analysis programs. GRID is limited by its representation of the fragments since it does not allow prediction of fragment orientation. A related program is HSITE which generates a map of the hydrogen-bonding regions of an enzyme active site, including the probability of hydrogen bond formation at each point.

To obtain accurate results from the programs discussed above, there is a need to understand, evaluate, and predict (or calculate) the binding free energy at the receptor sites. There also is a need to do so in a reasonable amount of computational time. The current predictive and short cut calculation methods leave much to be desired for improving structure-based drug design.

The prediction of binding free energy is according to the Expression:

Δg_{binding}=Δe_{binding}−TΔs_{binding}=Δe_{complex-formation}−TΔs_{complex-formation}+e_{solvation-desolvation}−TΔs_{solvation-desolvation} (1)

where,

Δg_{binding}=The total binding free energy for a protein-ligand complex.

Δe_{binding}=The interaction energy minus any intramolecular strain induced on complex formation.

Δs_{binding}=The change in conformational freedom induced by the formation of the complex.

Δe_{complex formation}=The interaction energy for the ligand/protein complex.

T Δs_{complex formation}=The change in entropy due to the reduction in flexibility in both the ligand and the protein upon complex formation.

Δe_{solvation-desolvation}=The energies of solvation are the energetic factors from the transfer of hydrophilic and lipophilic groups from an aqueous solvent to the more lipophilic region of the protein binding site.

Δs_{solvation-desolvation}=The entropy of solvation relating to the changes in the order of the solvent at the interface between ligand and solvent, and the protein and solvent on complex formation.

The ability to rapidly and accurately compute binding affinity of ligands for macromolecular receptors remains a problem in de novo structure-based drug design. Even though free energy perturbation calculations can produce relative binding free energies to within 1 kcal/mol, these methods are limited. The CPU (“Central Processing Unit”) time required to ensure convergence is excessive even using the fastest computers and the method is confined to small mutations between that pair of molecules for which there is an attempt to compute the difference in binding free energy.

To emphasize this point, in de novo structure-based drug design, there is the need to be able to test as many molecules as possible at proposed receptor sites in a short period of time and rank the tested structures based on an accurate prediction of the binding free energy. If small organic molecules are thought of as simple combinations of functional groups, candidate molecules may be considered as a choice and an arrangement, for example, of five functional groups. As such, a database of fragments that is being used to construct such a small organic molecules can result in an extremely large number of structures that must be evaluated. For example, even a very small database of 50 fragments will produce nearly 1 billion candidates of 5 functional groups −50⁵ combinations. As the size of the database of molecular fragments increases, it is readily seen that the number of possible combinations will increase dramatically. As such, the computational time necessary to test every combination, using current technology, is unreasonable and, therefore, not practical. The present invention provides a system and method that enhances the ability to conduct searches. In particular, a quick and accurate free energy estimation method is used for testing only the most meaningful combinations.

Conventional computational methods, such as those already discussed, use algorithms that hopefully overcome some of the computational burdens. However, in attempting to overcome the computational burdens, accuracy in predicting the binding free energy, which is a very important parameter to know in ranking possible candidates, has suffered greatly. For accurate estimates of binding free energy, sophisticated simulations using empirical potential and stepwise estimation of the changes in enthalpy and entropy at each stage of the thermodynamic cycle have been used to calculate the free binding energy. These calculations require extensive computational time for each molecule, which is impractical for normal structure-based drug design. These conventional methods, therefore, use scoring techniques that provide a short list of possible candidates for complete thermodynamic determination, or chemical synthesis and experimental determination of binding free energy.

In many of the methods for approximating the full expression for binding free energy, the scoring is based solely on the interaction energy between the ligand and protein in the complex as the single most important contributor to free energy. In others, ranking may be based more on spacial complementarity than chemical complementarity. In these cases, the salvation contributions are taken as being an approximation of the surface area. In either of these two methods the scoring is incomplete, which adversely affects the accuracy of the rankings of the candidate molecules or ligands.

In de novo structure-based drug design, there have been a number of methods to try to generate and rank lead candidates for docking at receptor sites. In the past, however, accuracy in predicting free energy suffered when short-cut methods were used to determine binding free energy. Therefore, unless excessive computational time was used to calculate the binding free energy in a three-dimensional protein-ligand complex, there will be a great chance of inaccuracy. Since the ranking of candidates molecules and ligands is primarily based on binding free energy, the chance of improperly ranking the candidates is very high.

The computational time needed for sampling a large number and variations of structures to generate potential lead candidates is usually very large and unreasonable. Since it is highly desirable to sample as many structures in as short a period of time as possible and then rank them based on accurate prediction of their binding free energy, there has been a need for a system and method to grow lead molecules without the computational burden of the past but with greater accuracy in binding free energy prediction.

### SUMMARY OF THE INVENTION

An object of the present invention is to provide a novel system and method for structure-based drug design. Thus, in one aspect, the invention provides a method of de novo designing molecules that bind to a receptor site on a protein comprising the steps of:

(a) building a molecule in the receptor site comprising: adding successive random molecular fragments to an initial molecular fragment that is loaded into the receptor site, estimating the free energy of the molecule being grown after each addition of a molecular fragment, and orienting each successive molecular fragment as it is added to the receptor site such that the free energy estimate for the molecule may be higher than a lowest free energy estimate possible for the molecule;

(b) repeating step (a) to generate a collection of molecules grown in the receptor site, and ranking the collection of molecules according to increasing free energy estimates to identify high-ranking molecules;

(c) selecting one or more functional groups of a high-ranking molecule identified in step (b) as a single restart fragment and using the restart fragment to build a second-generation of molecules according to steps (a) and (b),

(d) minimizing the energy of a protein/ligand complex comprising the receptor site and a second-generation molecule using an empirical force field

(e) quantitatively measuring the empirical interaction energy of the second-generation molecules, and ranking the molecules, wherein a molecule of low interaction strength is ranked higher than a molecule of more negative interaction energy is ranked higher than a molecule of less negative or positive interaction energy;

(f) modifying high-ranking molecules from step (f) based on qualitative analysis of the molecules including determination of chemical viability, synthetic feasibility, solubility, and effect of the molecule on the structure of the protein, whereas such modification comprises: atomic and/or functional substitutions, initiating growth from a specific receptor site, inclusion of salt bridges or hydrogen bonds, and solubility-enhancing measures.

(g) repeating steps (c) through (f) until a molecule is built which is identified as high-ranking in both steps (e) and (f).

In one embodiment, the receptor site is selected from the group consisting of the following: Src-homology-3 domain, Src-homology-2 domain, MDM2 protein, CD4 protein, and carbonic anhydrase protein (particularly, human carbonic anhydrase II protein). Preferably, the empirical interaction energy comprises CHARMM interaction energy and the empirical force field comprises CHARMM.

Another object of the present invention is to provide a novel system and method for structure-based drug design that has a more accurate method for predicting the binding free energy at the protein-ligand complex.

A yet further object of the present invention is to provide a novel system and method for building candidate molecules or ligands for binding at a receptor site that uses a more accurate method for predicting binding free energy at the protein-ligand complex.

Another object of the invention is to provide libraries of ligand candidates that bind to a receptor site of interest that have been generated using a de novo structure-based design method.

These and other objects will be described in greater detail in the remainder of the specification, taken together with the attached drawings.

### BRIEF DESCRIPTION OF THE DRAWINGS

FIG. 1 shows a block diagram of the method employed by the system of the present invention.

FIG. 2 is a block diagram for the method of estimating binding free energy that may be used in the molecule growth method employed by the system of the present invention.

FIG. 3 is a flow diagram of the molecular growth method of the system of the present invention.

FIG. 4 shows an example of a protein receptor site with at least an H₂ molecule loaded in it.

FIG. 5 shows an example of a protein receptor site with a molecule being built in it.

FIG. 6 shows first generation molecules as ligand candidates for the specificity pocket of Src SH3 domain.

FIG. 7 shows second- and third- generation molecules as ligand candidates for the specificity pocket of Src SH3 domain.

FIG. 8 shows a candidate ligand, 7e, from FIG. 7 that is able to form three hydrogen bonds as well as a significant II-stack with both Tyr55 and Trp42: (a) molecular structure of the candidate; (b) licorice diagram of the ligand in the binding site showing the residues with which a strong ligand should make interactions; (c) space-filling model showing the II-stacking with Tyr55 and Trp42; and (d) another view of the space-filling model.

FIG. 9 shows first- (b-e), second- (f-k) and third- (l-n) generation molecules as ligand candidates for the LP pocket of Src SH3 domain. The peptide PLPP that occupies the LP pocket is represented by “a.” The novel peptide molecule is represented by “o”; various side chains, R, are shown in FIG. 10.

FIG. 10 shows molecule, 9o, of FIG. 9, with various side chains as candidate ligands for the LP pocket of Src SH3 domain.

FIG. 11 shows a candidate ligand for the LP pocket of Src SH3 domain: (a) molecular structure of the ligand; (b) licorice diagram of the ligand in the building site showing the residues with which a strong ligand should make interactions; (c) space-filling model; and (d) another view of the space-filling model.

FIG. 12 shows the correlation between coarse-grained knowledge-based potential data and experimental binding constants in a series of ligands for the specificity pocket of Src SH3 domain. The experimental binding constants are plotted on the log scale since the logarithm of the binding constant is proportional to the experimental binding free energy.

FIG. 13 shows first-generation molecules as ligand candidates for CD4. Based on the free energy estimate and empirical interaction energies, these seven molecules are the best of 1000 molecules generated in the binding site.

FIG. 14 shows second-generation molecules as ligand candidates for CD4. The molecules generated were manually manipulated to improve qualitative and quantitative characteristics, including increasing II-stacking interaction, adding a bridge from the flexible chain connecting to the pyridine group to the sugar-like ring of carbon, and substitution of carbon for the oxygen atom on the seven-membered ring.

FIG. 15 shows a candidate ligand for the Phe43 binding pocket of CD4: (a) molecular structure of the candidate; (b) licorice diagram of the ligand in the binding site showing the residues with which a strong ligand should make interactions; (c) space-filling model of the ligand; (d) diagram showing the protein as a space-filling model and the ligand as a licorice diagram.

### DETAILED DESCRIPTION OF THE INVENTION

The present invention is a system and method for computational de novo structure-based drug design that employs a novel method for discovery and building of ligands, and a more accurate method for predicting binding free energy. Accordingly, the system and method of the present invention provide a better predictive de novo structure-based drug design tool by using a coarse-graining model with corresponding knowledge-based potential data. Moreover, in light of the use of the coarse-graining model, the novel molecular growth method of the present invention uses a metropolis Monte Carlo selection process for molecule growth that builds the molecules or ligands that result in a low free energy structure, but not necessarily the lowest free energy structure. Yet such a structure that is grown has a more accurate prediction of binding free energy and can be an acceptable drug design candidate.

The molecular growth method of the present invention uses the Metropolis Monte Carlo method to quickly search and sample the configuration space of the binding site. This is done with knowledge of the interactive potential for the fragments that are part of a database. The Metropolis Monte Carlo method also gives the system and method of the present invention the ability to identify very quickly fragments that will be useful in building the molecules or ligands.

Coarse-graining is a procedure commonly used in statistical mechanics to allow one to focus attention on events at an intermediate length scale so that one can deduce general trends without being overwhelmed by the variations in the most minute details. Formally, coarse-graining results in an averaging of the interaction potential within a space of a particular size. The size of these spaces should correspond to some physical distances in the system so that one can be assured of the essence of the details subsumed in the averaged potential. As applied to protein-ligand interactions, for instance, coarse-graining entails choosing a length scale that corresponds roughly with the distance over which a molecule can induce order within an aqueous solvent.

Using coarse-graining, a radius of contact between atoms of a protein and a ligand is defined and, when examining a database of crystal structures of protein-ligand complexes or evaluating the binding interactions in the course of design, atoms within a radius of contact (otherwise called an interaction radius) are considered to be in contact with one another. One advantage of the coarse-graining approach is that it integrates the solvent entropy terms of ligand binding into its potential surface. Another advantage of coarse-graining is that the potential surface is smoothed by the local averaging. This allows the space of possible molecules to be searched very efficiently by a Monte Carlo growth algorithm.

A knowledge-based potential is a set of interaction parameters that measure the contribution of various types of contacts to the free energy estimate. These parameters are derived from a database of structures by collecting statistics on the frequencies with which contacts are formed between all the various atom types. In combination with coarse graining, the knowledge-based potential provides a system for estimating binding free energies based on physical statistical inference.

The coarse-graining model with knowledge-based potential data follows from the application of the principles of canonical statistical mechanics to subsets of proteins. In particular, the model includes the determination that small subsets of a folded protein are in thermal equilibrium with each other. As such, the present invention employs these principles. Thus, there is a reasonable basis to contend that the information present in the crystal structures of proteins and crystal structures of protein-ligand complexes may be disassembled into constituent parts and the contribution of each part to the binding free energy may be assigned on the basis of probability. This permits the present invention to achieve the more accurate results for binding free energy predictions and applying them in the building of molecules or ligands.

According to the system and method of the present invention, the identification of candidate molecules is not just a search for the lowest free energy complex, but is a search to identify candidate molecules or ligands that form low free energy complexes at the receptor site and gives the best lead for drug design. This is accomplished using the growth method that employs a metropolis Monte Carlo selection process. The molecular growth method results in low free energy candidates generated of a desired length.

The present invention is a system and method for molecular growth through structure-based drug design that uses a novel method for building candidate molecules or ligands, and libraries of ligand candidates, and which employs a more accurate method to predict the binding free energy of the molecules or ligands as they are grown. The system and method of the present invention also allows the growth of candidate molecules or ligands in which there is a more accurate prediction of the binding free energy in a reasonable amount of computational time.

The method of the present invention allows one to quickly assess interactions to build a strong binding ligand and continuously provides suggestions for alterations and extensions of molecules that result in excellent chemical and spatial complementarity with the protein binding site. In this regard, the method of the present invention provides a quantitative score based on coarse-graining model with knowledge-based potential data (hereinafter such a quantitative score will be referred to as “free energy estimate score”) which is related directly to and is an approximation of the experimentally found binding free energy so that changes to a candidate molecule can be assessed quantitatively. Another advantage is that the method allows for interactive building of molecules. For instance, the structure-based design allows for the use of partially grown molecules as restart fragments and for the insertion of a specific interaction known to exist in naturally-occurring ligands (such as a salt bridge or II-II interactions), both of which affect the direction in which a candidate molecule is grown. Other advantages over methods of the prior art include efficiency of time, ability to generate and evaluate whole molecules rather than separate fragments which later require linkage, and ability to correlate the scoring method to known free energies of binding.

Unless indicated otherwise, the following definitions will apply in describing the present invention:

Binding is a physical event in which a ligand is associated with a receptor site in a stable configuration.

Docking is a computational procedure whose goal is to determine the configuration that will permit binding.

De novo structure-based drug design is meant to refer to a process of dynamically forming a molecule or ligand which is conducive to binding with a particular receptor site using knowledge of the protein structure.

Heavy Atom refers to any non-hydrogen atom within a molecule.

Ligand is a molecule that will bind with a target receptor.

Ligand candidate is a ligand proposed as a potential ligand. When a ligand candidate has been demonstrated experimentally to bind with a target receptor, it is redesignated a “ligand.”

Lead compound is a ligand that has been demonstrated experimentally to possess potential drug, therapeutic, and/or pharmaceutical uses.

Molecule refers to a combination of atoms bound together to form the smallest unit of matter of a molecular compound.

Rotamer (in the context of structure-based design) is a molecule that has been oriented in a binding site by torsional rotation of the bond between the molecule and the binding site.

Fragment is an atom or functional group used in the construction of a ligand or molecule.

Restart fragment is an atom or function group from a partially or wholly grown molecule, instead of a fragment selected at random from a library of fragments, that is used as the starting input for construction of a ligand or molecule using the method of the present invention,

Interaction radius is the maximum distance within which a protein and ligand atoms interact to bind to one another.

Reference state is an artificial configuration of ligand and protein atoms in which the frequency of all interaction types is exactly average. It is best described by the average probability \overline{p}=<pᵢⱼ>.

Quasichemical approximation is a mathematical method for converting probabilities to energies based on the principles of canonical statistical mechanics.

Empirical interaction energy refers to a formulation, in terms of mathematical equations and parameters, of the physical energy of interaction between a ligand candidate and a protein, wherein the specific form of the equations are adapted from studies of simpler interacting systems.

The term “alkyl” as employed herein refers to straight and branched chain aliphatic groups having from 1 to 12 carbon atoms, preferably 1-8 carbon atoms, which may be optionally substituted with one, two or three substituents. Unless otherwise specified, the alkyl group may be saturated, unsaturated, or partially unsaturated. As used herein, therefore, the term “alkyl” is specifically intended to include alkenyl and alkynyl groups, as well as saturated alkyl groups. Preferred alkyl groups include, without limitation, methyl, ethyl, propyl, isopropyl, butyl, tert-butyl, isobutyl, pentyl, hexyl, vinyl, allyl, isobutenyl, ethynyl, and propynyl.

As employed herein, a “substituted” alkyl, cycloalkyl, aryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, amino, alkylcarboxamido, arylcarboxamido, aminoalkyl, alkoxycarbonyl, carboxy, hydroxyalkyl, alkanesulfonyl, arenesulfonyl, alkanesulfonamido, arenesulfonamido, aralkylsulfonamido, phosphorylalkylcarbonyl, cyano, and alkylaminocarbonyl groups.

The term “cycloalkyl” as employed herein includes saturated and partially unsaturated cyclic hydrocarbon groups having 3 to 12, preferably 3 to 8 carbons, wherein one or two ring positions may be substituted with an oxo group, and wherein the cycloalkyl group additionally may be optionally substituted. Preferred cycloalkyl groups include, without limitation, cyclopropyl, cyclobutyl, cyclopentyl, cyclopentenyl, cyclohexyl, cyclohexenyl, cyclohexanone, cycloheptyl, and cyclooctyl.

An “aryl” group is a C₆-C₁₄ aromatic moiety comprising one to three aromatic rings, which may be optionally substituted. Preferably, the aryl group is a C₆-C₁₀ aryl group. Preferred aryl groups include, without limitation, phenyl, naphthyl, anthracenyl, and fluorenyl. An “arylalkyl” group comprises an aryl group covalently linked to an alkyl group, either of which may independently be optionally substituted or unsubstituted. Preferably, the arylalkyl group is C₁₋₆alk(C₆₋₁₀)aryl, including, without limitation, benzyl, phenethyl, and naphthylmethyl. An “alkaryl” or “alkylaryl” group is an aryl group having one or more alkyl substituents. Examples of alkaryl groups include, without limitation, tolyl, xylyl, mesityl, ethylphenyl, and methylnaphthyl.

A “heterocyclic” group is a ring structure having from about 3 to about 8 atoms, wherein one or more atoms are selected from the group consisting of N, O, and S. The heterocyclic group may be optionally substituted on carbon with oxo or with one of the substituents listed above. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino.

In certain preferred embodiments, the heterocyclic group is a heteroaryl group. As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, between one and about three heteroatoms selected from the group consisting of N, O, and S. Preferred heteroaryl groups include, without limitation, thienyl, benzothienyl, furyl, benzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

In certain other preferred embodiments, the heterocyclic group is fused to an aryl or heteroaryl group. Examples of such fused heterocyles include, without limitation, tetrahydroquinoline and dihydrobenzofuran.

As herein employed, the term “acyl” refers to an alkylcarbonyl or arylcarbonyl substituent.

The term “acyloxy” refers to an alkyloxycarbonyl or aryloxycarbonyl group.

The term “amido” as employed herein refers to formylamino, alkylcarbonylamino, or arylcarbonylamino. The term “amino” is meant to include NH₂, alkylamino, arylamino, and cyclic amino groups.

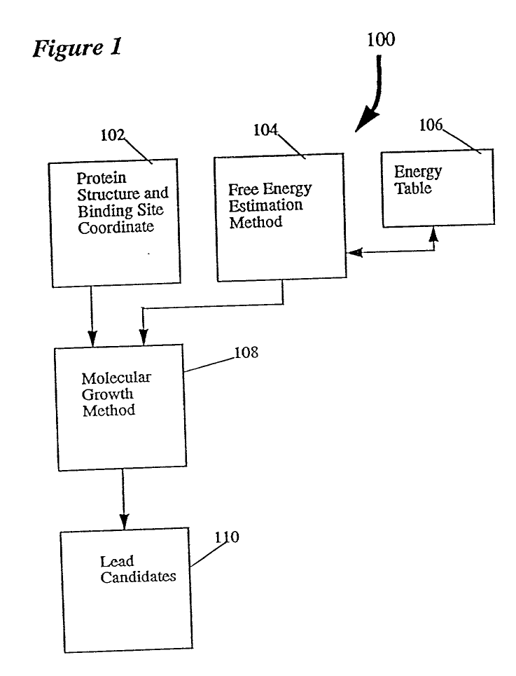

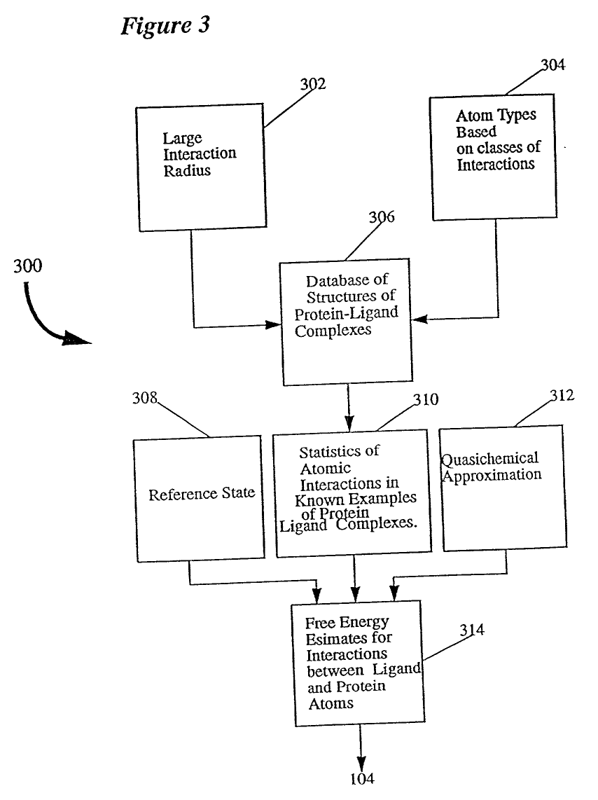

FIG. 1, generally at 100, provides a general schematic for the method of the present invention for building and ranking lead candidates for de novo structure-based drug design. As shown in FIG. 1, molecular growth method 108 receives inputs from 102 and 104.l At 104, information regarding the 1) protein structure and 2) its binding site is provided. This information includes the coordinate of the binding site, protein atom coordinates, and protein atom types in a standard Brookhaven Protein Data Bank (“PDB”) format or notation. With regard to the binding site, at least one coordinate is provided to identify a proposed receptor site. The free energy estimate method at 104 provides the estimate of free energy of the molecule or ligand that is being built. The free energy method receives input from energy table 106 which is developed according to FIG. 3, which will be described subsequently. Once a number of molecules or ligands have been built, they are ranked at 110, usually based on their binding free energy, as lead candidates for drug design.

The second input to molecular growth method 108 is the free energy estimate, 104, for the molecule being built. As stated, this free energy estimate method uses the information from the calculated free energy database developed according to FIG. 3. This prediction is based on proper selection of an interaction model and reference state, selection of an appropriate interaction radius, knowledge of the atom types at issue, information with regard to known structures of protein-ligand complexes (knowledge-based potential data), and quasichemical approximations.

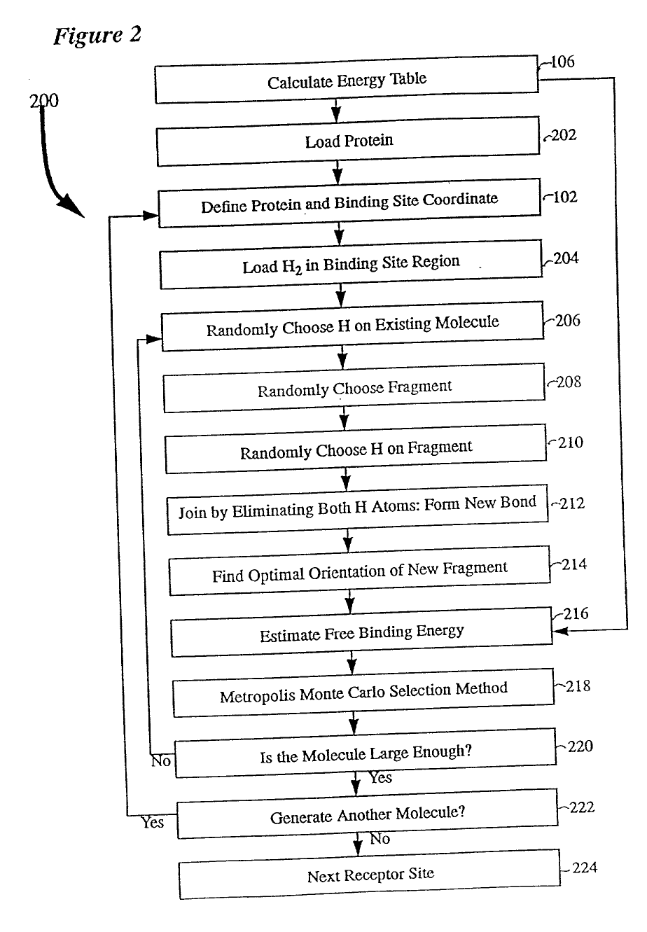

The molecular growth method at 108 is set forth in detail in FIG. 2, generally at 200, and will be described subsequently. This method employs a Metropolis Monte Carlo (“MMC”) selection process to control the acceptance of intermediate and finished molecules or ligands as conditions based on their estimated binding free energy.

Referring to FIGS. 1 and 2, the molecular growth method at 108 will be discussed in detail. At 106 in FIG. 2, the energy table (based on FIG. 3) is calculated. This table is calculated once for a given set of parameters. When the parameters are changed, the table is recalculated. For example, if the interaction radius is changed from 5 Å to 3 Å, the table may be recalculated. It is necessary to calculate the table at or near the beginning of the method of the present invention so that it may be accessed in building molecules or ligands.

At 202 in FIG. 2, the protein with a target receptor site is loaded. This is followed by step 102 at which the information about the protein that has the target receptor is input from 108 of FIG. 1. This information describes the protein structure and the binding site at issue. In describing the binding site, its relative position is given in the form of a coordinate. From this point, the molecular growth method will grow a molecule or ligand that is a simple organic molecule which consists of fragments joined with single bonds.

At 204, a hydrogen molecule (“H₂”) is positioned randomly in the binding site of the protein at the coordinate. This H₂ molecule is considered to be the existing molecule to satisfy step 206. According to step 206, one of the H atoms is randomly selected to be the site of the new bond.

Step 208 is performed by selecting at random a fragment from a library of fragments. For example, a library could include the fragments set forth in Table 1:

TABLE 1
Fragments For Molecule Growth Method

| | | | |
|---|---|---|---|
| 1. amide | 14. cyclohexane | 27. indole | 39. purine |
| 2. amine | 15. cyclohexane | 28. iodide | 40. pyramidine |
| 3. carbonyl | 16. cyclohexane | 29. methyl | 41. pyradine |
| 4. carboxylic acid | 17. cyclohexene | 30. n-butyl | 42. pyrrole |
| 5. chloride | 18. 1,2-dithian | 32. naphthalene | 43. sulfate |
| 6. cyanide | 19. ethane | 33. nitrile | 44. sulfide |
| 7. cyclo-octane | 20. ethene | 34. nitro | 45. tert-butyl |
| 8. cyclo-octane (2) | 21. fluoride | 35. phenyl | 46. tetrahydro- |
| 9. cyclo-octane (3) | 22. furan | 36. phosphate | furanyl |
| 10. cyclo-pentane | 24. glucose | 37. propane | 47. tetrahydro- |
| 11. cycloheptane | 26. hydroxyl | 38. propene | thienyl |
| | | | 48. thiophene |
| | | | 49. trifluoro- |
| | | | methyl |

At step 210, there is the random selection of at least one H atom on the randomly selected fragment. The selected H atom from the H₂ molecule and the H atom from the fragment will form the first fragment bond for the molecule being built at the protein binding site.

At step 212, the first (and new) bond is formed between the first fragment and the remaining H atom from the H₂ molecule loaded at step 204. As this bond is formed, the two H atoms that were selected are eliminated. By following the method of the present invention, it is assured that the new bond angles and bond lengths are reasonable approximations.

The next step is at 214 where the new fragment is oriented with respect to the bond just created and binding site by torsional rotation about the new bond so that the new fragment is properly situated at the binding site at a low energy level. Preferably in orienting the new fragment by torsional rotation about the new bond, the orientation is performed in fixed increments. These fixed increments are the smallest that will still permit reasonable computational times. Most preferably, the torsional orientation takes place in 60° increments. Orientations that are not sterically hindered are evaluated for their free energy value at step 216. Most preferably, the orientations are such that atom pairs are within 70% of the sum of their van der Waals radii. The position of the fragment or rotamer that yields low energy or the lowest energy is considered as a candidate for the molecule that is being grown.

Using the molecular growth method of the present invention, it is found molecules or ligands of any desired size may be built. This is realized by the ability to add fragments by the displacement of H atoms and binding of the atoms that were formerly connected to the displaced H atoms. The branching effect is fully realized in that any H atom of the structure that is attached to the protein is a potential growth site.

At 216, there is the prediction of free energy for the molecule being built. The method of estimating free energy is shown in detail in FIG. 3 at 300. FIG. 3 is a block diagram of the method for creating or calculating the energy table that is used to provide the desired free energy information for the molecule that is being grown fragment by fragment. In order to understand what is being estimated, it is necessary to understand the factors that determine the binding free energy of molecules. The remainder of the molecular growth method will be discussed before the method of estimating binding free energy is discussed in detail.

After step 216, the molecular growth method advances to step 218. Using a coarse-graining model with knowledge-based potential data, the system and method of the present invention evaluates the combinatorial search space, (the binding site of the protein), which is a rough energy landscape, to develop and identify candidate lead molecules that have a low, not necessarily the lowest, free energy complex. The system and method of the present invention overcome the multiple minimum problem, by using a MMC selection process at step 218. In order to determine whether this fragment will be accepted as a condition, once the binding free energy has been computed for each of the orientation of the rotamer, the MMC selection process at 218 makes a comparison with respect to the energy per atom before the current growth step and after the growth step at the optimal orientation. If there is a decrease in the energy per atom with the new built step, then that orientation is accepted as a condition. If an increase in energy per atom is experienced, however, it also is accepted as a condition but with a probability defined by Example (2):

$$p = exp [ - \frac{Δ g}{T} ] \qquad ( 2 )$$

where,

p=Probability of acceptance.

Δg=ΔG/N is the change in free energy per atom (with ΔG being the free energy difference upon adding the fragment at issue and N being the total number of atoms in the ligand).

T=Temperature.

The MMC selection process according to the system and method of the present invention allows the energy per atom to increase occasionally. This is needed if a small molecule is grown into a tight steric region of the binding site and the molecule had to be grown into the solvent or other unoccupied region and only marginally interact with the protein was present. Thus, allowing such an increase may provide an opportunity for a subsequent larger decrease in the free energy of binding.

Using the MMC selection process of the present invention as an optimization method has advantages. This method creates a low energy molecule in light of the random selection of rotamers that does not lead to significantly more metropolis failures. Further, the method will indirectly result in the tightest possible steric complementarity.

The step at 220 is to determine if the molecule that has been built is large enough at this point in time. If it is, then the method is directed to step 222. At step 222, it is determined if another molecule is to be built at this same binding site of the protein. If no additional molecules are built, the method will cease generating molecules for this protein binding site and the method will go to step 230 to wait for a next protein for which a candidate molecule or ligand is to be built. If the answer at step 222 is “yes” and another molecule is to be generated, then the method is directed back to step 204 where H₂ is added to the binding site of the protein where the new molecule or ligand is to be built.

If at 220 it is determined that the molecule is not large enough, then the method of the present invention returns to step 206 where another fragment is randomly selected for the addition to the existing molecule in the described manner.

In performing the method of the system of the present invention, consideration is given to the parameters surrounding performance of the method. Some of the parameters that are considered are the temperature at which the building takes place, the nearest approach allowed between atoms when assessing steric hindrance, and the angular increment in choosing fragment rotamers.

Preferably, the temperature that is selected is one that generates the largest number of low energy structures per unit of time. The nearest approach of two atoms is a percentage of the sum of their van der Waals radii since this will provide a good correlation with the nearest approaches in the database. The selected incremental amount for torsional orientation of the fragments can be further refined to smaller increments. However, such smaller increments may result in significantly more computational time to obtain results. Given the preferred parameters of 60° torsional rotation increments, 70% van der Walls contact radius, and an algorithmic temperature of 10.0, a molecule of at least twenty heavy atoms can be generated in about one second.

The method for estimating binding free energy will now be discussed in detail referring to FIG. 3. As stated, Expression (1) is used to estimating binding free energy:

Δg_{binding}Δe_{binding}−TΔs_{binding}=Δe_{complex-formation}−TΔs_{complex-formation}+Δe_{solvation-desolvation}−TΔs_{solvation-desolvation} (1)

Again, referring to FIG. 3, in estimating the binding free energy that will be part of the energy table, the items at 302 and 304 are combined with the items at 306 to generate the statistics of atomic interactions of known protein-ligand complexes, which is shown at 310. The information at 310 forms the knowledge-based potential interaction data that is used for estimating binding free energy of particular molecule being built.

At 302, a large interaction radius for the atoms that are to be bound in the protein-ligand complex is selected. A large interaction radius is selected because it will permit the solvation entropy effects to be accounted for. The most feasible length is selected for the interaction radius.

At 304, information is provided regarding the atom types based on the different classes of possible interaction. Table 2 provides examples of information that is included at 304:

TABLE 2
Atom-type Interaction Data

| Description | Atom Type | Code |
|---|---|---|
| Lipophilic Carbon Atoms | Fatty | CF |
| | Carbon | |
| Oxygen That Accepts Hydrogen Bonds | Oxygen | OA |
| | Acceptor | |

Item 306, therefore, includes at least information about the atom-types that are suspected will be present at the protein-ligand complex.

Now referring to the database at 306, this database includes a listing of structures of protein-ligand complexes that are known, their coordinates, and corresponding chemical elements. This database is one that can be continually updated as more information is obtained. The method of the present invention was applied to protein-ligand complexes for which structural and binding information has been previously determined experimentally. A sample of the protein-ligand complexes for which information is available in the Brookhaven Protein Data Bank (“PDB”) is shown in Table 3. Such examples include purine nucleoside phosphorylase (“PNP”) and amino acid binding protein (“LST”).

TABLE 3
Protein-Ligand Complex Database

| Protein | PDB Code |
|---|---|
| Purine Nucleoside Phosphorylase | 1 PNP |
| Amino Acid Binding Protein | 1 LST |

The information from 302 and 304, when combined with information at 306, result in the knowledge-based data at 310. Specifically, the information at 310 is the statistics of atomic interactions in known protein-ligand complexes which will permit a more accurate prediction of binding free energy for the molecule or ligand being built.

The next step is to compile the statistics at 310 into a set of interaction parameters that constitute the free energy contribution of interactions between specific atom types. Given the probability of atomic interactions in known protein-ligand complexes from 310, this information, along with reference state for the molecule or ligand being built, are combined. This reference state is selected such that it accounts for the solvent energy and configuration entropy effects.

The third item that is combined to generate the estimate from binding energy is an approximation of the protein-ligand complex of the molecule or ligand being built that is at 312. Preferably, the approximation utilized is a quasichemical approximation.

When the information from 308, 310, and 312 is combined, the results is the energy table at 106 that is used to provide the estimated free energy contributions for each type of interaction possible for the molecule or ligand being built. An example of the data provided in the free energy table at 106 is shown in Table 4:

TABLE 4
Energy Tables

| Protein Atom | Ligand Atom | Δgij |
|---|---|---|
| CF | CF | −2.18 |
| CF | OA | −1.13 |
| OA | CF | −0.89 |

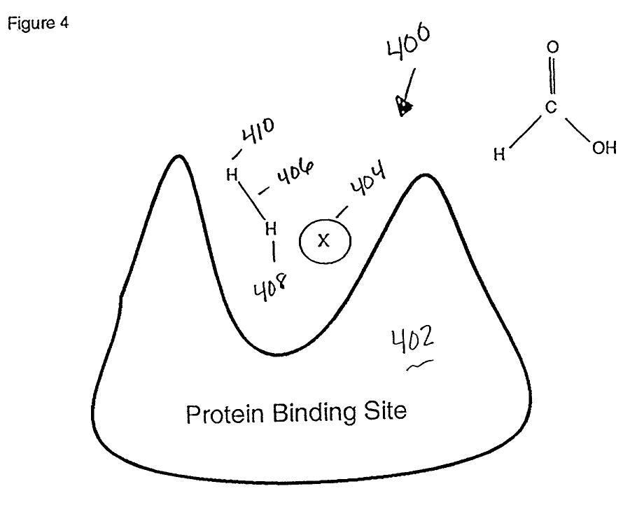

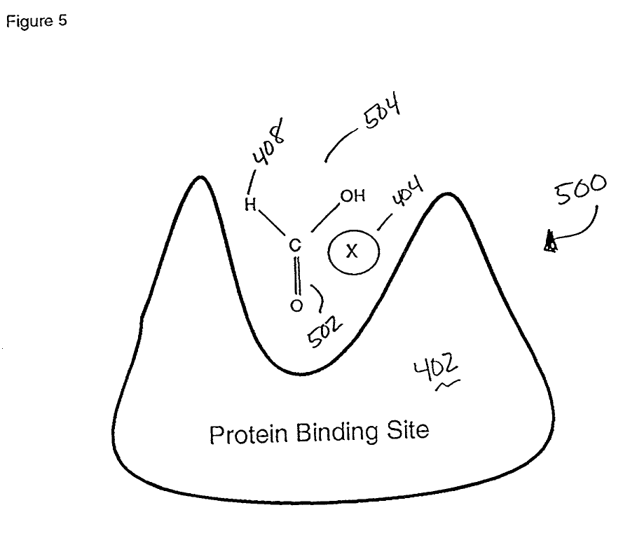

Before discussing the statistical support for the present invention, FIGS. 4 and 5 will be described. Those Figures graphically demonstrate aspects of FIGS. 1, 2, and 3. Referring to FIGS. 2 and 4, when protein 402, shown generally at 400, is loaded, it is defined and the coordinate of the binding site, such as coordinate “X” at 404, is determined. Next H₂ molecules, such as H₂ molecule 406, is added to the binding site. As would be understood, more than one H₂ molecule may be added to a single binding site.

After the H₂ molecule is loaded in the binding site, either H atom 408 or 410 is selected for forming the new bond. In this case, H atom 410 is selected. Once the selection is made, a fragment is randomly chosen from the fragment library and an H atom of that fragment is selected for establishing the new bond.

Referring to FIGS. 2 and FIG. 5, generally at 500, one H atom of fragment 502 is selected for forming the first bond When this bond is formed, the selected H atoms of H₂ molecule 406 (FIG. 4) and fragment 502 are eliminated. When new bond 504 is formed, the first fragment has been added in building the molecule.

The added fragment is now incrementally rotated about bond 504 to obtain the best fit and the free energy estimation is made based on the different orientations. An evaluation of the new molecule is made based on the MMC selection method described previously, then the remainder of the method of FIG. 2 is carried out.

As stated, the present invention, implements a coarse-graining model with corresponding knowledge-based potential data. This model is used because of the ability to apply the principles of canonical mechanics to subsets of folded proteins that are in thermal equilibrium with one another. As such, the crystal structures of proteins and crystal structures of protein-ligand complexes may be disassembled into constituent parts. The contribution of each part may then be assigned on the basis of probability.

Generally, in statistical mechanics any two states at the same energy have an equal probability of the occupation of a location. This is expressed in Expression (3):

$${P}_{ij}^{e} = \frac{exp [ - \frac{{e}_{ij}}{{kT}_{e}} ]}{Z} \qquad ( 3 )$$

where,

p^{e}ᵢⱼ=The energetic probability of an interaction between a protein atom of type i and ligand atom of type j.

eᵢⱼ=The energy of the interaction of a protein atom of type i and ligand atom of type j.

k=Boltzmann Constant.

Tₑ=Experimental temperature at which the data base information is obtained.

Z=Normalization Constant.

In forming protein-ligand complexes, configurations that have exactly the same energy are not equally likely to be formed. This is because two entropic effects arise from the strong presence of a boundary in the space sampled by the ligand. The entropic effects are: 1) solvent ordering (at the protein-solvent interface, ligand-solvent interface, and complex-solvent interface) and restrictions on the atomic interactions due to steric hindrance; and 2) the nearly fixed chemical structures of the protein and ligand. The entropic effects are not correlated to the energetic events, and, therefore, may be expressed as the sampling probability p^{s}, which relates to the entropic effects, as set forth in Expression (4):

$${P}_{ij}^{s} = \frac{exp [ - \frac{{s}_{ij}}{k} ]}{Z} \qquad ( 4 )$$

where,

p^{s}ᵢⱼ=The sampling probability of an interaction between a protein atom of type i and ligand atom of type j.

sᵢⱼ=Entropy of interactions between a protein atom of type i and ligand atom of type j.

k=Boltzmann Constant.

Z=Normalization Constant.

The product of p^{e} from Expression (3) and p^{s} from Expression (4) gives a relationship between the total probability and gross binding free energy (that is dependent on the interaction model chosen to describe the atomic interactions between the protein and ligand in the protein-ligand complex). This is shown in Expression (5):

$${P}_{ij} = {P}_{ij}^{e} {P}_{ij}^{s} = \frac{exp [ \frac{{e}_{ij} - {Ts}_{ij}}{{kT}_{e}} ]}{Z} = \frac{exp [ \frac{{g}_{ij}^{*}}{{kT}_{e}} ]}{Z} {p}_{ij} where , \qquad ( 5 )$$

where,

p^{s}ᵢⱼ=The total probability of interactions between a protein of type i and ligand atom of type j.

p^{e}ᵢⱼ=The energetic probability of an interaction between a protein atom of type i and ligand atom of type j.

p^{s}ᵢⱼ=The sampling probability of an interaction between a protein atom of type i and ligand atom of type j.

eᵢⱼ=The energy of the interaction of a protein atom i and ligand j.

sᵢⱼ=Entropy of interactions between a protein atom of type i and ligand atom of type j.

k=Boltzmann Constant.

Tₑ=Experimental temperature at which the data base information is obtained.

Z=Normalization Constant.

The interaction model that is chosen is comprised of an interaction radius and a set of eligible atom types.

Expression (5) can be changed so that it is solved for g*ᵢⱼ, the free energy as set forth in Expression (6). This comprises the quasichemical approximation. Expression (6) is determined from the frequency of observed interactions:

g*ᵢⱼ=−kTₑ log(pᵢⱼ)−kTₑ log(Z) (6)

where,

g*ᵢⱼ=The gross free energy of an interaction between a protein atom of type i and ligand atom of type j.

Tₑ=Experimental temperature at which the data base information is obtained.

k=Boltzmann Constant.

Z=Normalization Constant.

pᵢⱼ=The total probability of an interaction between a protein of atom type i and ligand atom of type j.

When the appropriate reference state is selected, the Normalization Constant can be eliminated. The reference state contributes a free energy of \overline{g} to every gross free energy term gᵢⱼ*. This is shown via Expressions (7), (8), and (9):

\overline{g}=−kTₑ log(\overline{p})−kTₑ log(Z) (7)

gᵢⱼ=gᵢⱼ*−\overline{g} (8)

$${g}_{ij} = - {kT}_{e} log [ \frac{{P}_{ij}}{\overline{p}} ] \qquad ( 9 )$$

where for Expressions (7), (8), and (9),

\overline{g}=Gross free energy of the reference state.

\overline{p}=The average probability of an interaction between protein and ligand atoms.

gᵢⱼ=Net free energy of the interaction between a protein atom of type i and ligand of type j.

g*ᵢⱼ=The gross free energy of an interaction between a protein atom of type i and ligand atom of type j.

pᵢⱼ=The total probability of an interaction between a protein of atom type i and ligand atom of type j.

Tₑ=Experimental temperature at which the data base information is obtained.

k=Boltzmann Constant.

Z=Normalization Constant.

Expression (9) relates the statistical information about interatomic interactions in the crystal structures of the protein-ligand complex to term by term contributions to binding free energy. The gᵢⱼ's, when summed, will approximate the complete form of free energy in Expression (1) that is provided at 104.

The correct interaction model and reference state for application of a knowledge-based potential data to a protein-ligand binding event is deducted from the terms in Expression (6) (repeated below:):

g*ᵢⱼ=−kTₑ log(pᵢⱼ)−kTₑ log(Z) (6)

More specifically, changes in the solvation entropy with regard to the complex formation occur because of a gain or loss in the solvent order, which is a correlation between the potential surface of the solvent exposed to atoms in the ligand, protein, or complex. These correlations are generally twice the size of a water molecule beyond the boundary of the ligand, protein, or complex. As the interactions between the ligand and protein result in desolvation, there is a change in the configurational entropy. Thus, to form a particular intermolecular contact between a protein and ligand, each of the atoms in contact must be desolvated. Where there has been a large amount of order destroyed by this process, there is an entropic increase due to the desolvation for the formation of that particular contact.

The selection of the large interaction radius, between the protein and ligand, should be the correlation length of solvent ordering. When this is the case, the probability of the specific contacts observed occurring will include the average effect of the contribution of solvation entropy to the prediction of free energy. As such, the system and method of the present invention will select a large interaction radius for the interaction model. For the purposes of the present invention, the interaction model will define a ligand atom to be in contact with a protein atom if they are within the interaction radius of one another.

Each contact formed involves an energy loss based on desolvation. This must be accounted for in the reference state. In defining the reference state, the specificity of the loss due to desolvation of a specific contact becomes a general element that is factored in and the remaining energetic contributions in the interaction model with a large interaction radius take into account simply that a loss due to desolvation has taken place.

In selecting the reference state, there effectively is unrestricted spatial sampling of the ligand with respect to the protein. As such, the reference state has no perceived notion of the chemical structure. Thus, the difference between the free estimates of a specific structure and that of the reference state accounts for the loss of configurational entropy in the collection of atoms upon formation of a specific, largely rigid chemical structure. If Expression (8) is now considered

gᵢⱼ=g*ᵢⱼ−\overline{g} (8)

where \overline{g} is subtracted from g*ᵢⱼ, the entropic effect of unrestricted sampling and the effect of desolvation is taken into account.

Using the Expressions set forth above, the system and method of the present invention score, and then rank, the candidate structures based, in large part, on the determination of the total binding free energy that is defined by Expression (10):

$$Δ G = {\sum }_{ij} {g}_{ij} {Δ}_{ij} \qquad ( 10 )$$

where,

ΔG=The total binding free energy.

gᵢⱼ=Net free energy of the interaction between a protein atom of type i and ligand of type j.

Δᵢⱼ=This term is zero unless i and j are within the interaction radius of each other, in which case, it is equal to one.

Using the interaction model and reference state that was previously discussed, ΔG is an approximation to the complete change in free energy in the complex formation. Coarse-graining that was discussed included the entropic effects of solvation and the reference state has taken into consideration the effects of solvation energy and the configurational entropy. Moreover, according to the system and method of the present invention, a closer look is taken of the atom types and their affect on the energy contributions that really take place in complex formation, not a generalization of the atom types.

The following Examples will further illustrate the invention. The Examples are not intended, and should not be interpreted, to limit the scope of the invention which is more fully defined in the claims which follow.

### EXAMPLE 1

Correlation with Experimental Binding Energies

To test the accuracy of the approximation of binding free energy, the method of the present invention was compared with experimental measurements of binding free energy. The system of the present invention may be operated in one of three modes: automatic, directed, or assisted. In the automatic mode, all that is necessary is to provide the starting protein structure and a coordinate on the protein to specify the vicinity of the binding site. Based on this input, the system generates ligands with at least one atom within an interaction radius length of the specified coordinate. The directed mode is an interactive method in which the user specifies the molecular fragments which are selected and where they are to bind. The assisted mode begins with the user specifying a fragment. Then, the assisted mode proceeds automatically. This mode allows the user to incorporate a specific molecular fragment into the molecule being grown.

The method of the present invention was applied to several protein-ligand complex systems for which structural and binding information has been previously determined experimentally, including the following: purine nucleoside phosphorylase (“PNP”), and human immunodeficiency virus-1 protease (“HIV-1 protease”).

Purine Nucleoside Phosphorylase

The method for approximating binding free energy according to the present invention was applied to guanine based ligands that had been designed, synthesized, and assayed for purine nucleoside phosphorylase (“PNP”). PNP ligands comprising any of the X or R groups listed in Table 5, were interactively built using the system and method of the present invention in a directed mode in a low energy conformation. As such, the molecules were tested as if they had been generated using the de novo molecular growth method of the present invention. In other words, given enough computation time the system and method of the present invention would have generated the listed molecules and any corresponding conformation. Undirected generation, i.e. not in a directed mode, of these exact ligands is highly improbable. Generation of the PNP ligands shown in Table 5 by the system and method of the present invention allowed for the correlation of free energies taken as the logarithm of the binding constants or IC₅₀ measurement, which is an experimental determination of the propensity for complex formation, to knowledge-based potentials of the present invention. The same approach was used for the SH3 domain and HIV-1 protease examples.

The following PNP ligands were tested by the method of the present invention:

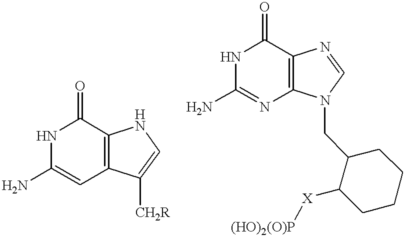

where R and X comprise the following groups listed in Table 5, respectively.

TABLE 5
PNP Candidate Ligands

| | | | free energy |
|---|---|---|---|
| | | Kᵢ or IC₅₀ (μM) | estimate |
| | Phosphate | (1 mM | per heavy |
| R | sensitivity | phosphate) | atom |
| 2-hydroxyphenyl | low | 0.27 | −18.1 |
| 2-tetrahydrofuranyl | low | 0.07 | −16.2 |
| 2-tetrahydrothienyl | high | 0.011 | −16.6 |
| 2-thienylmethyl | low | 0.021 | −16.6 |
| 3-methoxyphenyl | low | 0.082 | −18.1 |
| 3-methylcyclohexyl | high | 0.025 | −18 |
| 3-thienylmethyl | low | 0.025 | −15.8 |
| 3-trifluoromethylcyclohexyl | high | 0.025 | −13.2 |
| 3-trifluoromethylphenyl | low | 0.036 | −12.3 |
| 4-hydroxyphenyl | low | 0.26 | −18.7 |
| cycloheptyl | high | 0.03 | −17.1 |
| cyclohexyl (no methylene) | high | 1.3 | −17 |
| cyclohexyl | high | 0.047 | −17.4 |
| cyclopentyl | high | 0.029 | −18 |
| methylphenyl | low | 0.057 | −19.4 |
| phenyl | low | 0.051 | −18.7 |
| pyridin-3-yl | low | 0.025 | −18.5 |
| | | Kᵢ or IC₅₀ (μM) | SMoG |
| | phosphate | (1 mM | energy per |
| X | sensitivity | phosphate) | heavy atom |
| -(CH₂)₂- | low | 0.035 | −17.8 |
| -(CH₂)₃- | high | 0.62 | −18.8 |
| -O(CH₂)₂- | high | 1 | −18.9 |
| GMP | low | 530 | −14.1 |
| GDP | low | 360 | −13.9 |
| | | | free energy |
| | | Kᵢ or IC₅₀ (μM) | estimate |
| | Phosphate | (1 mM | per heavy |
| R | sensitivity | phosphate) | atom |
| GTP | low | 490 | −14.7 |
| dGMP | low | 300 | −14.4 |
| gGDP | low | 37 | −14.9 |
| dGTP | high | 32 | −14.4 |
| acyclovir | low | 200 | −15.8 |
| acyclovirMP | low | 6.6 | −14.4 |
| acyclovirDP | high | 0.009 | −14.4 |
| acyclovirTP | high | 0.31 | −14.4 |

Each molecule contains a guanine or 9-deazaguanine fragment, which was held fixed at the coordinates in the 1 ulb crystal structure of guanine. The binding mode of the balance of the structure was determined by conformational search on the potential surface provided by the coarse-grained knowledge-based potential of the present invention. The molecules that were marked as having low phosphate sensitivity are those whose binding constant changes by a factor of 15 or less upon increase of the concentration of phosphate to 50 mM. The highly sensitive molecules are affected in some instances by a factor of 140.

The knowledge-based potential data, according to the present invention, correlates well with the experimental binding free energy for over five orders of magnitude in the binding constants. The strong correlations that were found for the binding free energy predictions according to the system and method of the present invention indicate an ability to effect de novo drug design of lead molecules.

HIV-1 Protease

HIV-1 protease has been the target of a wealth of structure-based drug design efforts. See Abdel-Meguid, S. S. et al., Biochemistry 1994, 33:11671-11677. Thompson, S. K.; Murthy, K. H. M.; Zhaong, B.; Winborne, E.; Green, D. W.; and Fisher, S. M. et al., J. Med. Chem. 1994 37:3100-3107. However, in choosing a system of ligands for proofing the correlation between course-grained knowledge-based potential and experimentally determined binding free energies, several considerations needed to be applied. First, the experimental determinations had to have been performed under identical conditions among the members in the system. Secondly, it was required for the binding constants to span a wide range. Thirdly, it was required for the binding mode coordinates to be published or attainable via conformational search. Fourth, it was necessary for the molecules to be structurally diverse and yet of roughly the same molecular weight. The following HIV-1 protease ligands were tested by the method of the present invention:

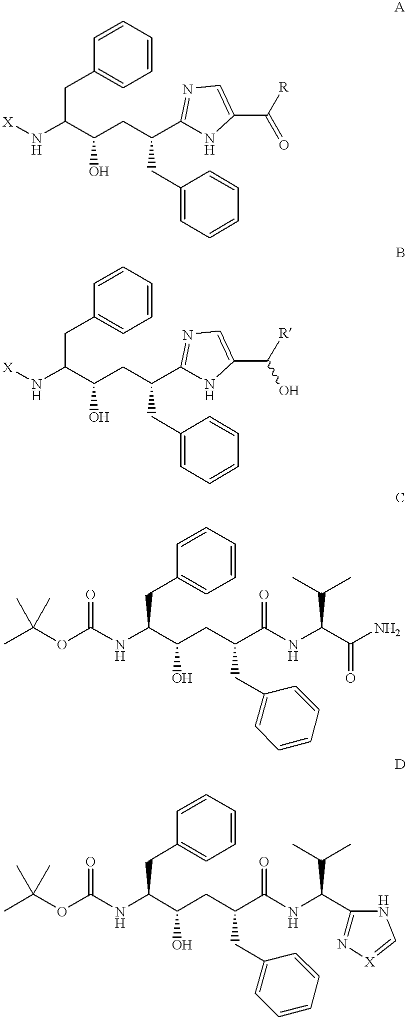

where, X and R (or R′) comprise the following groups listed in Table 10.

TABLE 10
HIV-1 Protease Candidate Ligands

| | | | | Free energy |
|---|---|---|---|---|
| | | | | estimate per |
| molecule | X | R (or R′) | Kᵢ (nM) | heavy atom |
| A | Boc | H | 3500 | −12.1 |
| A | Boc | Me | 370 | −12.5 |
| A | Boc | Et | 92 | −13 |
| A | Boc | n-Pr | 150 | −13.8 |
| A | Boc | i-Pr | 83 | −13.4 |
| A | Boc | CMe₂CHCH₂ | 270 | −13.6 |
| B | Boc | Me(R) | 13300 | −12.3 |
| B | Boc | Me(S) | 13300 | −12.4 |
| B | Boc | i-Pr(R) | 2700 | −13.3 |
| B | Boc | iPr(S) | 2700 | −13 |
| C | | | 1.4 | −13.3 |
| D | CH | | 18 | −14.1 |
| D | N | | 4.2 | −13.8 |

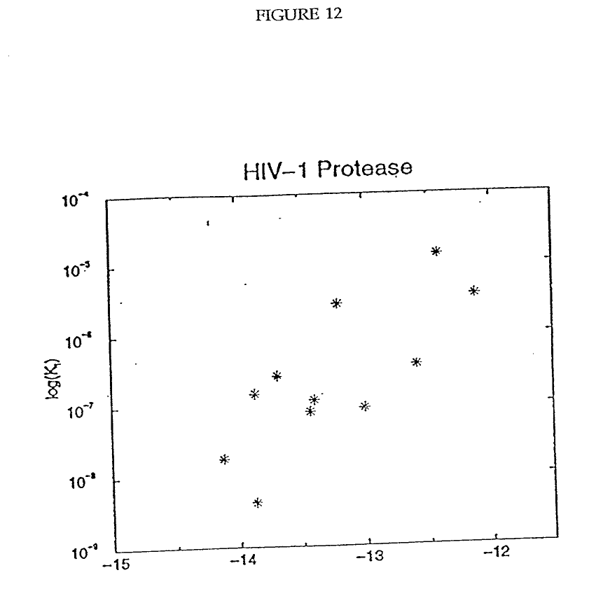

As in the PNP example above, these molecules share common structural motifs, so that by using the crystal structures (1 hps and 1 sbg) to define the coordinates of these motifs and using conformational search on the balance of each molecule, the binding mode of each ligand was determined. Also, the structurally specific waters in the binding site were included as part of the protein. Correlation between coarse-grained knowledge-based potential was determined using the structure-based design method of the present invention and experimental binding constants (CHARMM interaction energies) is shown in FIG. 12.

Table 11 provides a summary of the quantitative correlations for the three protein-ligand systems for which structural and binding information has been obtained experimentally: PNP, SH3 domain, and HIV-1 protease. In each case, significant correlation was observed. The probably of random occurrence was defined as the probability that a random selection of the same number of points would have the given correlation constant. Thus, the confidence that the observed correlations were systematic and not the result of sparse sampling are 99.8%, 88.9%, and 95.0% for the PNP, SH3 and the HIV-1 systems, respectively.

TABLE 11
Summary of Correlation Data

| | correlation | | probability of |
|---|---|---|---|
| system | coefficient | no. of data points | random occurrence |
| PNP | 0.8 | 17 | 0.002 |
| SH3 | 0.81 | 8 | 0.11 |
| HIV | 0.77 | 11 | 0.05 |

### EXAMPLE 2

De Novo Design

The de novo design method of the present invention can generally be discussed as occurring in five stages as follows:

I. Guided Tour of the Binding Site—Generate many molecules and observe the qualitative features that arise with a high frequency among those that score best. Note the potential interactions types that are favored.

II. Growth from a Template—Select some parts of molecules from the previous observations to use as restart fragments. Using the method of the present invention, generate molecules from specific sites on these fragments. This allows the user to select the direction of molecular growth and attempt to include the features observed above.

III. Quantitative Analysis—Use an empirical force field such as CHARMM to minimize the energy of the complex formed with each of the best molecules from stage II. Those molecules that score well with both the free energy estimate provided by the method of the present invention (otherwise referred to herein as coarse-grained potential) and the empirical interaction energy are scrutinized further.

IV. Qualitative Analysis—These high scoring molecules are scrutinized on the basis of qualitative interactions, chemical viability, synthetic feasibility, solubility, as well as observation of the structural changes the ligand induces in the protein.

V. Optimization—Utilize the method of the present invention and chemical intuition to enhance the quantitative and qualitative score of the best molecules. Generally, this involves atomic and/or functional substitutions, growth from a specific site, or inclusion of salt bridges or hydrogen bonds, or efforts to increase the solubility of the molecule.

It is the object of this invention to provide a method of de novo designing molecules that bind to a receptor site on a protein comprising the steps of:

(a) building a molecule in the receptor site comprising: adding successive random molecular fragments to an initial molecular fragment that is loaded into the receptor site, estimating the free energy of the molecule being grown after each addition of a molecular fragment, and orienting each successive molecular fragment as it is added to the receptor site such that the free energy estimate for the molecule may be higher than a lowest free energy estimate possible for the molecule;

(b) repeating step (a) to generate a collection of molecules grown in the receptor site, and ranking the collection of molecules according to increasing free energy estimates to identify high-ranking molecules;

(c) selecting one or more functional groups of a high-ranking molecule identified in step (b) as a single restart fragment and using the restart fragment to build a second-generation of molecules according to steps (a) and (b);

(d) minimizing the energy of a protein/ligand complex comprising the receptor site and a second-generation molecule using an empirical force field

(e) quantitatively measuring the empirical interaction energy of the second-generation molecules, and ranking the molecules, wherein a molecule of low interaction strength is ranked higher than a molecule of more negative interaction energy is ranked higher than a molecule of less negative or positive interaction energy;

(f) modifying high-ranking molecules from step (e) based on qualitative analysis of the molecules including determination of chemical viability, synthetic feasibility, solubility, and effect of the molecule on the structure of the protein, whereas such modification comprises: atomic and/or functional substitutions, initiating growth from a specific receptor site, inclusion of salt bridges or hydrogen bonds, and solubility-enhancing measures.

(g) repeating steps (c) through (f) until a molecule is built which is identified as high-ranking in both steps (e) and (f).

Particularly, the invention provides a method of de novo designing molecules for binding to a receptor site present on a substrate, wherein the substrate is preferably selected from the group consisting of: Src-homology-3 domain, Src-homology-2 domain, MDM2 protein, CD4 protein, and carbonic anhydrase protein.

It is another object of the invention to build libraries of ligand candidates which bind to a receptor site of interest according to a de novo structure-based design method, wherein the method comprises:

(a) evaluating a receptor site for a molecular make up of at least a portion of the receptor site to which a molecule being grown will bind and generating at least a coordinate of at least a portion of the receptor site to which the molecule being grown will bind, and outputting, at least with respect to the molecular make up of the receptor site, the coordinate of the portion of the receptor site to which the molecule being grown will bind;

(b) estimating free energy of the molecule being grown using knowledge-based potential data to estimate free energy and outputting the estimated free energy; and

c) building a molecule for binding to the receptor site using the outputs from steps (a) and (b), with the building step including building the molecule by selecting molecular fragments at orientations that will result in free energy estimates for the molecule that may be higher than a lowest free energy estimate possible for the molecule. In a preferred embodiment, libraries of ligand candidates are built which bind to a receptor site on the following substrates: Src-homology-3 domain, Src-homology-2 domain, MDM2 protein, CD4 protein, and carbonic anhydrase protein.

CD4

The CD4 protein is an immunoglobulin-family transmembrane coreceptor expressed in the helper T-cells. It participates in contact between the T-cells and antigen-presenting cells by binding to the nonpolymorphic part of the class II major histocompatibility complex (MHC-II) protein, which is followed by the activation of the bound Lck kinase which leads to downstream activation events in T-cells. The human immunodeficiency virus (HIV) gains entry into a T-cell binding protein gp120 to the CD4 receptor. This gp120 binding site in the vicinity of Phe43 of CD4 was the target for ligand design.

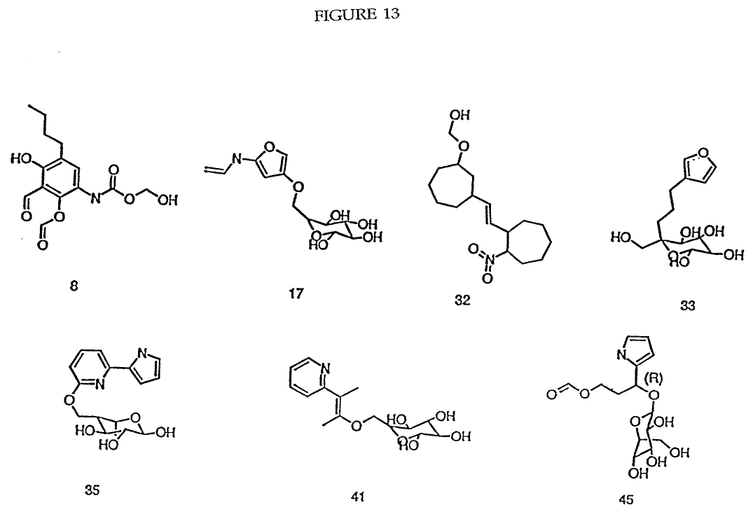

Among the possible interactions that arose in stage I of the design process, it was apparent that II-II interaction with the phenyl ring of the Phe43 was important, as well as formation of hydrogen bonds in the narrow pocket bounded by Lys 46 and Asp 56. The first generation of molecules resulting from the method of the present invention are shown in FIG. 13, where a hydrogen-bonding core and a hydrophobic moiety can be seen in the same relative orientation in most of the molecules. Results of a quantitative analysis of these first-generation CD4 candidates of FIG. 13 are shown in Table 12, which follows.

TABLE 12
Quantitative Analysis of the First-Generation CD4
Candidates Shown in FIG. 13

| | Free energy estimate score | CHARMM interaction |
|---|---|---|
| molecule | per heavy atom | energy (kcal) |
| 8 | −26.2 | −82.3 |
| 17 | −30 | −80.9 |
| 32 | −28.5 | −53.6 |
| 33 | −36.3 | −70.6 |
| 35 | −26.8 | −80.8 |
| 41 | −45.7 | −99 |
| 45 | −26.9 | −59.8 |

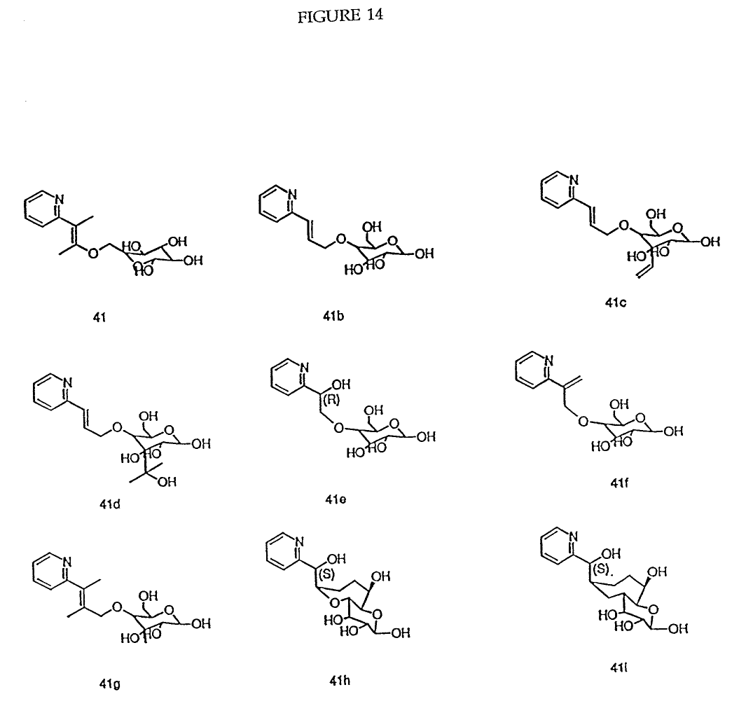

Using the quantitative data shown in Table 12 as well as qualitative features led to the selection of molecule 41 for further analysis. FIG. 14 shows second-generation molecules as ligand candidates for CD4. Molecule 41b was created by manually altering the point of attachment of the sugar-like ring structure of molecule 41, this improving II-stacking interaction with Phe42. Molecules 41c, 41d, and 41g were derived from 41b through ring substituents generated by the claimed design model. Molecule 41e was generated from 41b by shortening and saturating the flexible chain connecting it to the pyridine group, which also improved the II-stacking. Molecule 41f follows from 41e via manual alteration suggested by the geometry of the binding site. Molecule 41h was derived from 41e by adding a bridge from the flexible chain to the sugar-like ring which preserved the binding conformation of the molecule, thereby enhancing its rigidity. Molecule 41i was derived from 41h by manual substitution of carbon for the oxygen atom on the seven-membered ring; this substitution weakens the II-stacking due to its effects ib various angles in molecule 41i. Table 13 describes the quantitative and qualitative analysis of these second-generation molecules.

TABLE 13
Quantitative Analysis of the Second-Generation CD4
Candidates Shown in FIG. 14

| | Free energy | | | |
|---|---|---|---|---|
| | estimate score | CHARMM energies (kcal) | | |
| molecule | per heavy atom | interaction | strain | net |
| 41 | −45.7 | −99 | 14.4 | −84.3 |
| 41b | −47.9 | −139 | 20 | −119 |
| 41c | −45.1 | −128 | 20.7 | −107.3 |
| 41d | −46.1 | −120 | 33.2 | −86.8 |
| 41 | −50.6 | −112 | 36.6 | −75.4 |
| 41f | −50.5 | −116 | 20.1 | −95.9 |
| 41g | −49.4 | −82 | 15 | −67 |
| 41h | −49.9 | −119 | 22.5 | −96.5 |
| 41i | −48 | −86 | 20.5 | −65.6 |

The strain energy is calculated as the difference in internal energy between the bound conformation and the conformation resulting from gas phase minimization to convergence using the adapted-basis Newton-Raphson method. The net CHARMM energy is the interaction energy plus the strain energy.

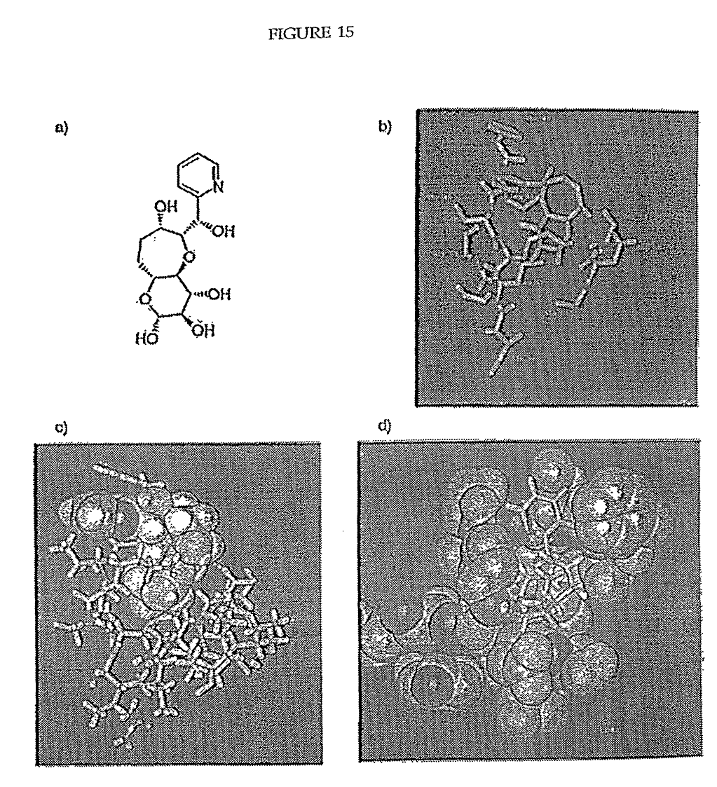

FIG. 15 shows the three-dimensional structure of molecule 41h in the gp120 building site of CD4. FIG. 15(a) shows the ligand candidate that binds to a receptor site on the CD4 protein generated de novo using a structure-based drug design method which comprises the following Formula V:

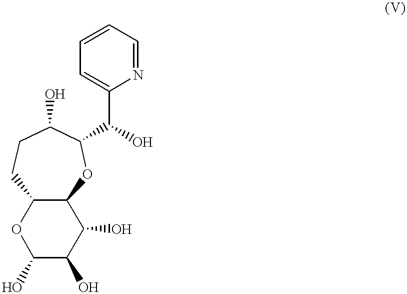

The interactions present within the ligand candidate include partial II-stacking with Phe43, as well as four intermolecular hydrogen bonds with Lys46 and Asp56 and one intramolecular hydrogen bond which stabilizes the orientation of the puridine group. The seven-membered fused-ring bridge gives the molecule a great deal of rigidity in its bound conformation.

SH3 Domain

The Src-homology-3 (SH3) domain is a conserved domain found in a variety of intracellular signal transduction mediators such as PI3K, Grb2, Crk, etc., and participates in the diversity of protein-protein interactions mediating the signal pathway eventually leading to the cell responses such as cell growth, differentiation, and migration. Irregularities in these processes may contribute to the cause of several common disease, thus making it important to consider the SH3 domain as a candidate for therapeutic inventions.

Two classes of polyproline helix peptide were recently found to bind the Src SH3 domain (class I RXLPPLP and class II LPPLPXR). They are accommodated in three pockets formed by conserved residues: the specificity pocket occupied by arginine, directing the peptide orientation, and two LP pockets each occupied by an LP pair. The following two design efforts focus on one LP pocket and the specificity pocket.

Specificity Pocket

Combinatorially synthesized small molecule ligands attached to the N-terminus of “biasing element” PLPPLP (occupying two LP pockets, part of class I peptide without Arg and with X=P) were recently shown to bind to the specificity pocket. Combs, A. P.; Kapoor, T. M., Feng, S., Chen J. K., Daude-Snow, L. F., Schreiber, S. L., J. Am. Chem. Soc., (1996) 118:287-288. The assay revealed an extremely strong selection for the first monomer attached to the N-terminal proline. Since the acylated monomer provided the opportunity for growth in the pocket, it was used as a restart fragment, and the method of the present invention was used to grow ligands into the specificity pocket by insisting that the growth proceed only from the acyl H atom on this monomer, thus preserving the peptide-like nature of the molecule.

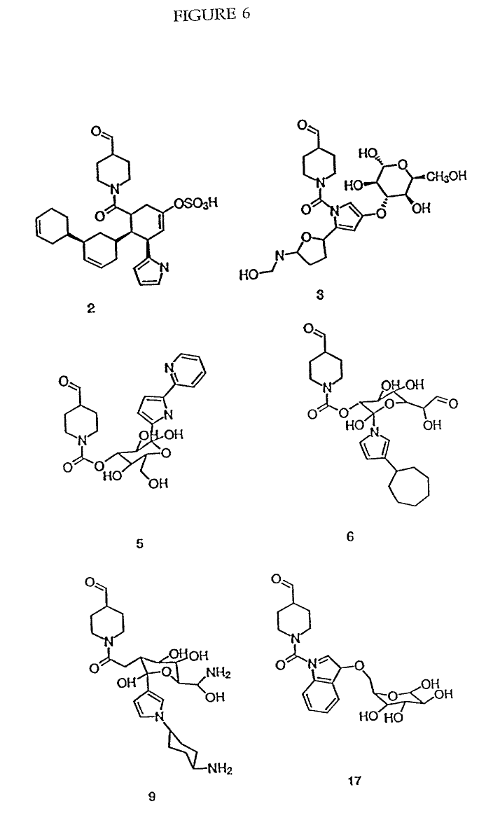

After stage I, it was apparent that two characteristics of high-scoring molecules were of special importance. First, the formation of a large amount of hydrophobic contacts with Tyr 55 and Trp 42. Second, the formation of hydrogen bonds with the donors and acceptors on Asp 23 and Thr 20. The first-generation ligands are shown in FIG. 6. The molecules shown in FIG. 6 are the best 6 of 100 molecules generated in the binding site using the acylated monomer as the restart fragment. These molecules possessed the following qualitative traits: a glucose-like ring that forms hydrogen bonds with residues in the RT loop of the pocket, and an unsaturated ring system with hydrophobic contacts with the tryptophan and tyrosine residues in the binding pocket. Molecule 3 scored well quantitatively (see Table 6) and also provided suggestions for improved hydrophobic interactions with Tyr55 and Trp42.

One basic template was selected for further optimization (molecule 3), in which a sugar group made the hydrogen-bonding interactions and the remainder of the molecule left a rich potential for enhancing the hydrophobic interactions. This selection was based predominantly on opportunities for enhancing the scoring of molecule 3 using the coarse-grained knowledge-based potential molecular growth method of the present invention, rather than CHARMM interaction energy, which, though strong, was far weaker than for other first-generation candidates, as shown in Table 6.

TABLE 6
Qualitative Analysis of the First-Generation SH3 Specificity Pocket
Candidates shown in FIG. 6

| | Free energy estimate score | CHARMM interaction |
|---|---|---|
| molecule | per heavy atom | energy (kcal) |
| 2 | −32.7 | −117.9 |
| 3 | −32.9 | −59.4 |
| 5 | −23.9 | −107.2 |
| 6 | −29.4 | −62.9 |
| 9 | −30 | −68.4 |
| 17 | −33.2 | −46.3 |

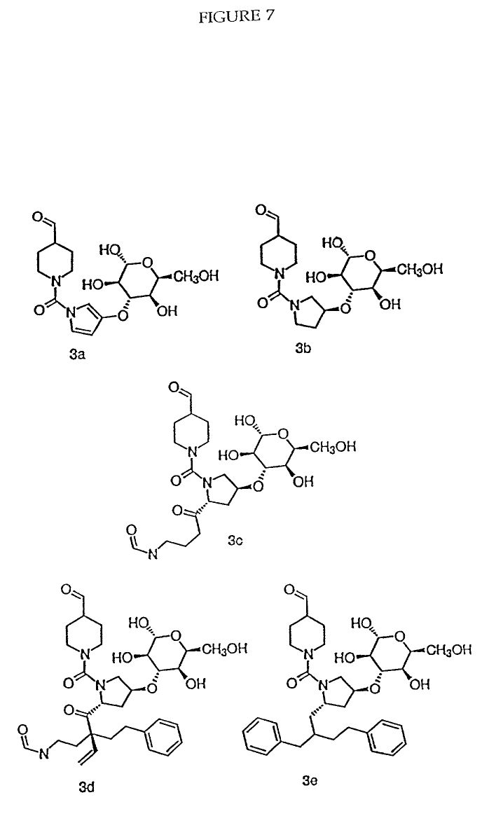

Some non-essential functionality was removed to prepare molecule 3a (see Molecule 3a is derived from molecule 3 of FIG. 7 by removing one substituent from the pyrrole ring. The considerable strain energy of molecule 3a was relieved by saturating the five-membered ring such that the conformation of the glucose was altered as little as possible.

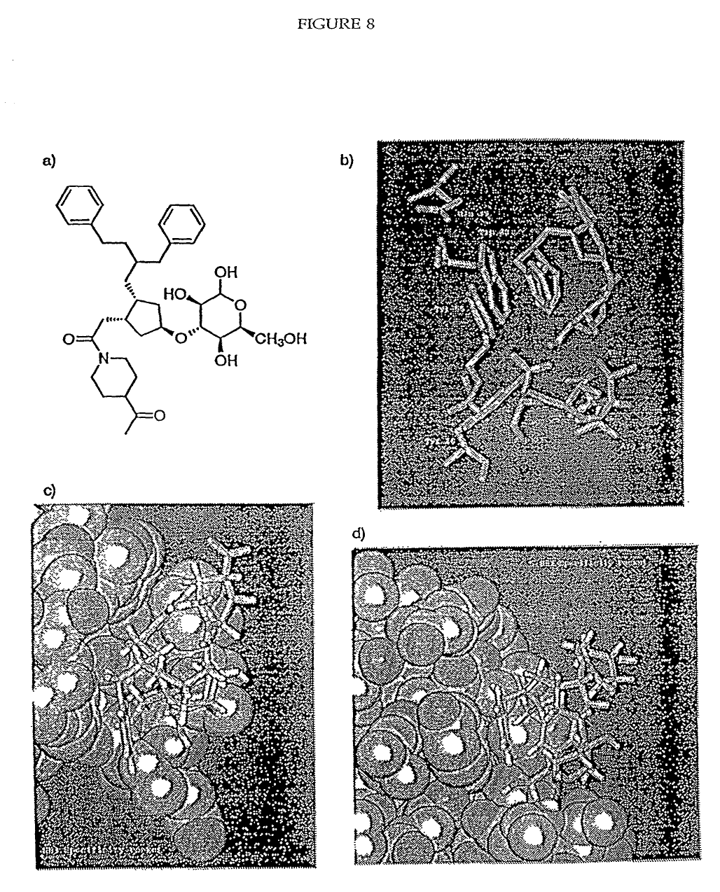

Saturation of the pyrrole group led to molecule 3b, the restart fragment for subsequent design, whose internal strain energy was greatly reduced in relation to molecule 3a, as shown in Table 7. By using a few hydrogen atoms on this molecule as sites for potential growth, the method of the present invention was used through two generations of optimization. After minimization of the complex structure with CHARMM, molecule 3b was used as the restart fragment, with only the H atoms on the central five-membered ring as eligible attachment points. The best scoring candidate, 3c, was used as a restart fragment to create molecule 3d, whose phenyl ring forms a U-stacking configuration with Tyr55. Molecule 3e was derived from molecule 3d by manual alteration after noting that the arrangement of the terminal amide group could form part of a phenyl group that made a partial II-stack with Trp42. Also, the joining chain was made more flexible by the elimination of one carbonyl group, converting the carbon from sp² to sp³, thus reducing internal strain energy. The resulting molecule, 3e, shown in FIG. 8, is able to form two II-stacking interactions and three hydrogen bonds with the protein.

TABLE 7
Quantitative Analysis of the Second-Generation
SH3 Specificity Pocket Candidates Shown in FIG. 7

| | Free energy | | | |
|---|---|---|---|---|
| | estimate score | CHARMM energies (kcal) | | |
| molecule | per heavy atom | interaction | strain | net |
| 3a | −27.3 | −54.6 | 32.2 | −21.6 |
| 3b | −27.1 | −51.4 | 18.5 | −32.9 |
| 3c | −33.4 | −77.2 | 27.4 | −49.8 |
| 3d | −34.7 | −59.7 | 24.4 | −35.3 |
| 3 | −37.8 | −57.9 | 19 | −38 |

The strain energy is calculated as the difference in internal energy between the bound conformation and the conformation resulting from gas phase minimization to convergence using the adapted-basis Newton-Raphson method. The net CHARMM energy is the interaction energy plus the strain energy.

SH3 Domain LP Pocket

The design effort for the LP pocket faced additional challenges from the desire to replace LP in position 2,3 of the biasing element with a mimetic. It was desired for the new ligand to possess amide bonds with the proline residues (1 and 4 of biasing element) at each boundary of the pocket, a goal which severely constrained the geometry of the molecules that would be reasonable structures.

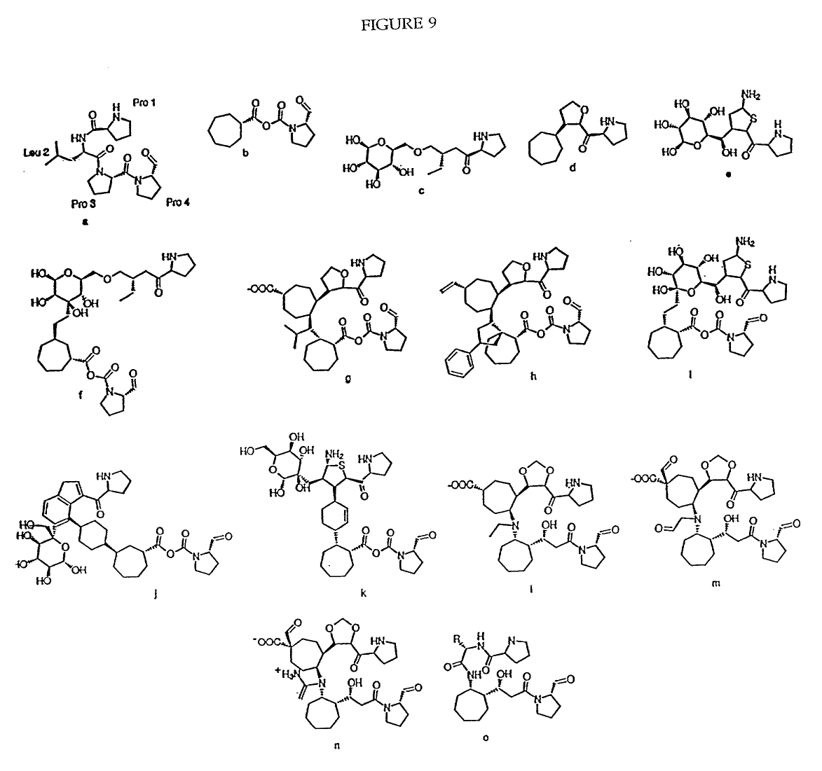

The method of the present invention successfully designed candidate ligands for the LP pocket by using proline 1,4 as restart fragments such that molecular growth proceeded inward toward the pocket from each bounding proline. These first-, second-, and third- generation ligand candidates for the LP pocket of Src SH3 domain are shown in FIG. 9.

The following are the results of stage I design: In place of Pro 3, the method of the present invention demonstrated a strong preference for a seven membered hydrophobic ring (FIG. 9b) grown from Pro 4 which makes hydrophobic contacts with Tyr 52, Arg 11, Tyr 8, and Pro 19 side chains. In place of Leu 2, the present method suggested several candidates grown from Pro 1, the best of which are shown in FIG. 9c-e. These first generation molecules revealed that in the region where Pro 3 was bound, the preference is mainly for hydrophobic fragments whereas the Leu 2 site prefers fragments which make both hydrophobic contacts (with Trp 34) and hydrogen-bonding interactions (with residues Asn 51 and Ser 50). This last feature is absent in the purely hydrophobic leucine side chain.

In order to combine the above segments, several linkers were built both manually and using the molecular growth method of the present invention using the above segments as restart fragments. The most appropriate of these (i.e., those that allowed covalent attachment without inducing much conformational strain energy) were used to join each pair of Leu site and Pro site fragments. The slight strains induced by linking were reduced with CHARMM minimization. The second generation molecules (i.e., the best linked molecules) are shown in FIG. 9f-k. Each of these molecules scored well under the structure-based design method of the present invention as well as empirical measurements as well as strong CHARMM scores. Quantitative results for the first three generations of SH3 LP pocket candidates are given below in Table 8.

TABLE 8
Quantitative Analysis of the First Three Generations
of SH3 LP Pocket Candidates Shown in FIG. 9

| | Free energy | CHARMM |
|---|---|---|
| | estimate score | interaction |
| Molecule | per heavy atom | energy (kcal) |
| 9a | −19.6 | −20.7 |
| 9b | −35.6 | −8.9 |
| 9c | −39.4 | −23.68 |
| 9d | −34.3 | −8.2 |
| 9 | −33.8 | −23 |
| 9f | −36.6 | −28.6 |
| 9g | −38.2 | −53.8 |
| 9h | −47.9 | −45.8 |
| 9i | −31.6 | −52.7 |
| 9j | −35.7 | −46.5 |
| 9k | −34.1 | −53.2 |
| 9l | −37.4 | −42.9 |
| 9k | −34.1 | −53.2 |
| 9n | −38.8 | −66.2 |
| 9o (R = H) | −28.6 | −28.3 |

Qualitative analysis of these molecules indicated that the phenyl ring of molecule 9h induces rather large deformation of the protein structure in the vicinity of the specificity pocket. Therefore, it was discarded. Those candidates containing glucose-like groups were also discarded in order to avoid molecules containing unnatural sugars. The only candidate that remained for further analysis was 9g. Manual optimization with respect to synthetic feasibility led to the third-generation molecules 9 1-n which also contained some additional hydrogen-bonding groups, contributing to stronger binding energies, as shown by the CHARMM interaction energies of Table 8.

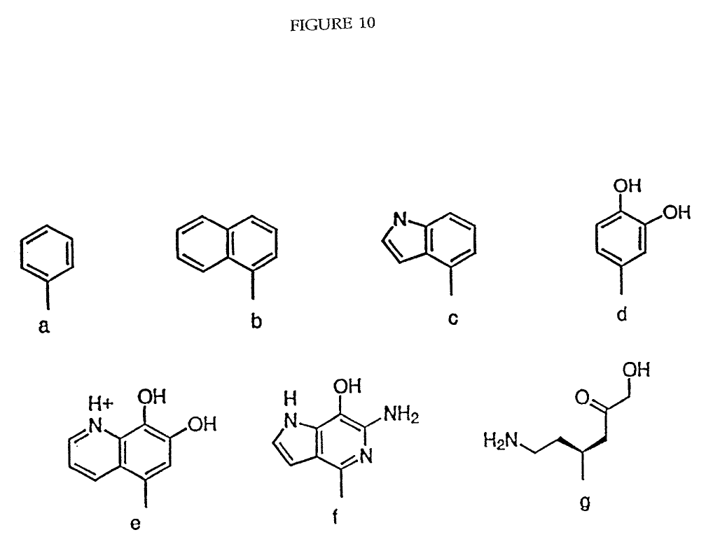

At this point, however, synthesis of the resulting Leu substitute would still be rather involved. Thus, in order to design the simplest molecule possible from the point of view of synthesis, while maintaining strong interactions, the seven membered ring in place of Leu3 was removed. It became apparent that an amino acid linker would provide the ideal joint between Pro 1 and the Pro 3 substitute (ideal in the sense of synthetic ease and linkage without inducing conformational stress; moreover, the side chain direction points into the Leu 2 site). It should be noted that this observation was the direct result of examining candidate molecules which had resulted from the de novo structure-based design method of the present invention. The side chain of this amino acid was built by the present method with growth restricted to position R on molecule 9o. Several aromatic side chains were generated (FIG. 10a-c) and further optimized manually by inserting various hydrogen- bonding fragments, yielding the fourth-generation molecules. Quantitative analysis of these final ligand candidates is shown in Table 9.

TABLE 9
Quantitative Analysis of the Fourth-Generation
SH3 LP for the LP Pocket Shown in FIG. 10

| | Free energy | | | |
|---|---|---|---|---|
| | estimate score | CHARMM energies (kcal) | | |
| molecule | per heavy atom | interaction | strain | net |
| 9a | −34 | −25.4 | 30.8 | +5.4 |
| 9b | −40.3 | −27.6 | 4.9 | −22.7 |
| 9c | −38.7 | −25.4 | 34.9 | +9.5 |
| 9d | −34.4 | −41.1 | 12.3 | −28.8 |
| 9 | −37.2 | −46.8 | 12.1 | −34.7 |
| 9f | −38.4 | −52.3 | 6.3 | −46 |
| 9g | −36.4 | −52.9 | 31.2 | −21.7 |

The strain energy is calculated as the difference in internal energy between the bound conformation with Pro 1 and Pro 4 fixed, and the conformation resulting from gas phase minimization to convergence using the adapted-basis Newton-Raphson method, also holding Pro 1 and Pro 4 fixed. In this sense, the strain energy is the energy difference upon binding the portion of the helical-substituted biasing element in consideration to the protein. The net CHARMM energy is the interaction energy plus the strain energy.

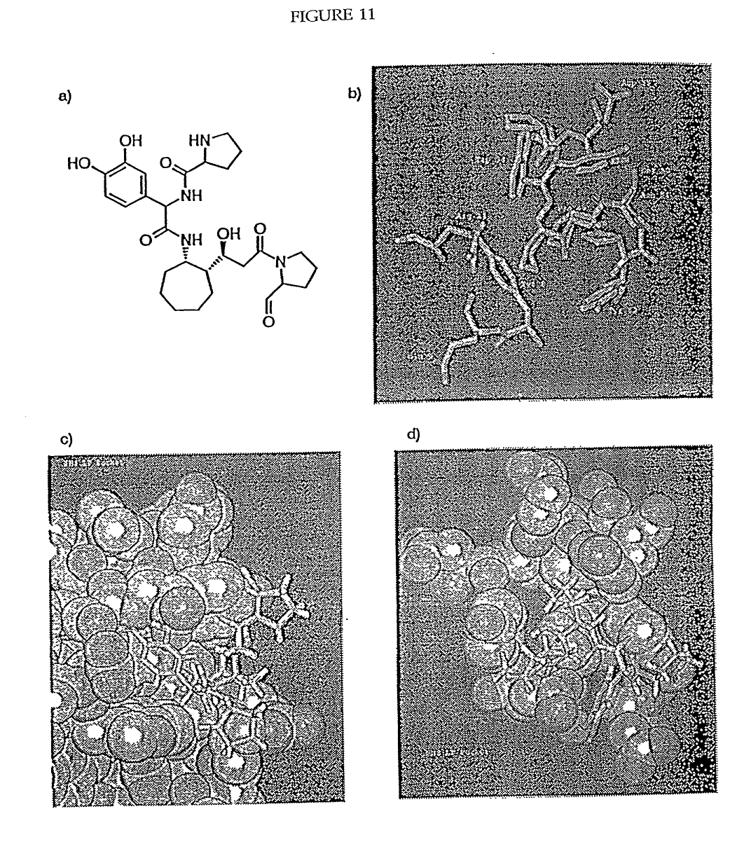

Molecule 10d of FIG. 10 is further described in FIG. 11 as an example of the best LP pocket ligand candidates designed using the (molecules 10d-g). As shown in FIG. 11, this molecule is able to form three hydrogen bonds and possesses significant hydrophobic and electrostatic complementarity while bridging the bounding proline residues of the biasing element.

SH2

The Src homology 2 (SH2) domain, like SH3, is a modular component present in many signal transduction proteins. It allows rapid formation of stable protein complexes and may also regulate protein function through intramolecular binding events. SH2 domains recognize phosphotyrosyl residues in a specific sequence context, while SH3 domains recognize a PxxP motif and additional residues that mediate binding specificity. Src homology 2 (SH2) domains are found in a variety of signalling proteins and bind phosphotyrosine-containing peptide sequences. SH2 domains mediate protein/protein interactions by binding phosphotyrosyl proteins with high specificity.

The design method of the present invention was used in automatic mode by inputting only the starting protein structure and a coordinate on the protein to specify the vicinity of the binding site to generate ligand candidates for the SH2 domain.

In one embodiment, the invention provides a library of ligand candidates generated using the method and system of the present invention for the SH2 domain having Formula I, II, or III:

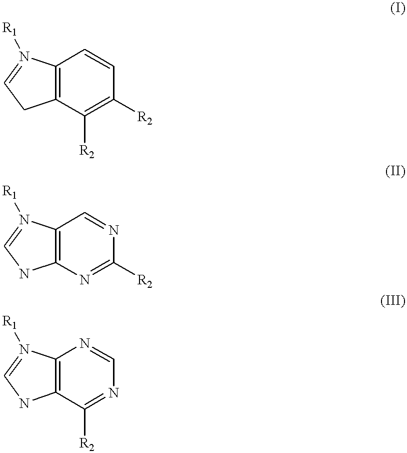

wherein

R1 is alkyl, aryl, heteroaryl, alkylaryl, arylalkyl, cycloalkenyl, cycloalkyl, cycloalkylamido, and arylalkylamido; and

R2 is independently at each occurrence selected from the group consisting of hydrogen, alkyl, aryl, heteroaryl, (heteroaryl)alkyl, alkylaryl, arylalkyl, cycloalkenyl, cycloalkyl, acyl, acyloxy, amino, amido, and alkoxy, wherein one or more groups may be optionally substituted.

With respect to ligand candidates which bind to the SH2 domain, the following preferred embodiments are applicable:

R1 is preferably selected from the group consisting of (C₆₋₁₀)ar(C₁₋₆)alkyl, preferably (C₆₋₁₀)ar(C₁₋₃)alkyl, more preferably benzyl; (C₃₋₈)cycloalkyl(C₁₋₆)alkyl, preferably (C₃₋₆)cycloalkyl(C₁₋₃)alkyl; (C₆₋₁₀)ar(C₁₋₆)alkylamido, preferably (C₆₋₁₀)ar-(C₁₋₃)alkylamido, more preferably benzylamido; and (C₃₋₈)cycloalkyl(C₁₋₆)alkylamido, preferably (C₃₋₆)cycloalkyl(C₁₋₃)alkylamido; wherein any of the aryl or cycloalkyl groups may be optionally substituted. In certain particularly preferred embodiments, the substituent is a phosphoryl group, and R1 is phosphoarylalkyl or phosphoarylalkylamido. Most preferably, R1 is selected from the group consisting of phosphobenzyl or phosphobenzylamido.

R2 is preferably selected from the group consisting of hydrogen; C₁₋₈alkyl, preferably C₁₋₆alkyl, more preferably C₄₋₆alkyl; C₆₋₁₄aryl, preferably C₆₋₁₀aryl; heteroaryl; (C₆₋₁₀)ar(C₁₋₆)alkyl, preferably (C₆₋₁₀)ar(C₁₋₃)alkyl; (heteroaryl)alkyl; (C₃₋₈)cycloalkyl, preferably (C₃₋₆)cycloalkyl, more preferably cyclopropyl, cyclopentyl, cyclopentenyl, cyclopentadienyl, cyclohexyl, or cyclohexenyl; C₂₋₈alkoxycarbonyl, preferably C₂₋₆alkoxycarbonyl, more preferably methoxycarbonyl, ethoxycarbonyl, or benzyloxycarbonyl; C₂₋₈acyloxy, preferably C₂₋₆acyloxy, more preferably C₂₋₄acyloxy, most preferably butylryloxy, butenoyloxy, propionyloxy, or propenoyloxy; C₁₋₆alkoxy, preferably C₁₋₄alkoxy; C₁₋₆alkylamido, preferably C₁₋₄alkylamido; and C₁₋₆alkylamino, preferably C₁₋₄alkylamino; any of which groups may be optionally substituted.

Most preferably, R2 is selected from the group consisting of pentyl, pentenyl, butyl, butenyl, phenyl, cyclopentyl, cyclopentenyl, cyclopentadienyl, cyclohexyl, butyryloxy, butenoyloxy, propionyloxy, propenoyloxy, propylamido, propenylamido, ethylamido, and ethenylamido.

MDM2

The tumor-suppressor p53 is a short-lived protein that is maintained at low, often undetectable levels in normal cells. The protein is expressed at very low levels in normal cells but accumulates in response to DNA damaging agents such as ultraviolet radiation. This increase is accompanied by transcriptional upregulation of the expression of a number of proteins including MDM2 which can in turn inhibit p53 dependent transcriptional activation, resulting in down-regulation of p53 activity. The MDM2 protein binds the transcriptional activation domain of p53 and blocks its ability to regulate target genes and to exert antiproliferative effects and p53 activates the expression of the MDM2 gene resulting in an autoregulatory feedback loop.

The development of small molecules that bind to the p53-binding pocket on the MDM2 protein poses a continuing challenge. Recently, researchers reported a gene encoding a small protein that binds tightly to the p53-binding pocket on the MDM2 protein. Botter, A. et al., Design of a Synthetic MdM2-binding Mini Protein that Activates the p53 Response In Vivo, Curr. Biol. 7(11):860-869 (Nov. 1, 1997). Using the method of the present invention, applicants have designed ligand candidates which bind to the p53-binding pocket on the MDM2 protein.

In another embodiment, the invention provides a library of ligand candidates generated using the method and system of the present invention which bind to the p53-binding pocket on the MDM2 protein having Formula IV:

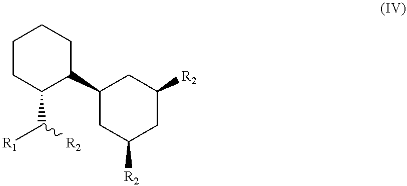

wherein

R1 is selected from the group consisting of hydrogen, alkyl, cycloalkyl, arylalkyl, aryl, heteroaryl, and (heteroaryl)alkyl, any of which groups may be optionally substituted; and

R2 is independently at each occurrence selected from the group consisting of hydrogen, alkyl, cycloalkyl, arylalkyl, aryl, heteroaryl, and (heteroaryl)alkyl, OH, O(R3), amino, wherein the aryl or heteroaryl group may be optionally substituted,

wherein R3 is selected from the group consisting of alkyl, cycloalkyl, aryl, aralkyl, (heteroaryl)alkyl, and heteroaryl, wherein the aryl or heteroaryl group may be optionally substituted.

With respect to ligand candidates which bind to the p53 binding pocket on the MDM2 protein, the following preferred embodiments are applicable:

In certain preferred embodiments, R1 is preferably selected from the group consisting of H; C₁₋₈alkyl, preferably C₁₋₆alkyl, most preferably methyl, ethyl, propyl, isopropyl, isobutyl, or butyl; C₃₋₈cycloalkyl, preferably cyclopropyl, cyclopentyl, or cyclohexyl; (C₆₋₁₀)ar(C₁₋₆)alkyl, preferably(C₆₋₁₀)ar(C₁₋₃)alkyl, more preferably benzyl; heterocyclic having one or more, preferably between one and about three, more preferably one or two, ring atoms independently selected from the group consisting of N, O, and S; heterocyclic(C₁₋₆)alkyl, preferably heterocyclic(C₁₋₃)alkyl; and C₆₋₁₀aryl, preferably phenyl; any of which groups may be optionally substituted.

In certain preferred embodiments, R2 independently at each occurrence is preferably selected from the group consisting of H; hydroxy; amino; C₁₋₈alkyl, preferably C₁₋₆alkyl, more preferably C₁₋₄alkyl; C₃₋₈cycloalkyl, preferably cyclopropyl, cyclopentyl, or cyclohexyl; (C₆₋₁₀)ar(C₁₋₆)alkyl, preferably(C₆₋₁₀)ar(C₁₋₃)alkyl, more preferably benzyl; heterocyclic having one or more, preferably between one and about three, more preferably one or two, ring atoms independently selected from the group consisting of N, O, and S; heterocyclic(C₁₋₆)alkyl, preferably heterocyclic(C₁₋₃)alkyl; and C₆₋₁₀aryl, preferably phenyl; any of which groups may be optionally substituted. In certain particularly preferred embodiments, R2 is selected from the group consisting of hydroxy and amine. In certain particularly preferred embodiments, R1 is selected from the group consisting of methyl, ethyl, and isopropyl.

Carbonic Anhydrase

Carbonic anhydrase is an enzyme that catalyzes the reaction between water and carbon dioxide to produce carbonic acid and hydrogen ions. Seven isozymes are known, and of these human carbonic anhydrase II, in particular, is of interest. Human carbonic anhydrase II protein causes increased intraocular pressure in the aqueous humour, which has been correlated to the development of the ocular disease, glaucoma. The enzyme is comprised of 260 amino acids, with a 15 angstrom deep pocket with Zn⁺² ion at the base, and is coordinated by three histidine residues.

To date, several strong drug inhibitors of human carbonic anhydrase II have been developed. Current efforts have focused on exploring benzene sulfonamide-based ligands. See Sigal and Whitesides, Bioorganic and Med. Chem. Ltrs, 6(5):559-564 (1996). Using the method of the present invention, the inventors designed a ligand candidate for human carbonic anhydrase II. Such candidate was then synthesized and shown to successfully bind to human carbonic anhydrase II.

The various technical and scientific terms used herein have meanings that are commonly understood by one of ordinary skill in the art to which the present invention pertains. As is apparent from the foregoing, methods known to those of ordinary skill in the art can be utilized in carrying out the present invention. Further, although the foregoing invention has been described in detail by way of illustration and example for purposes of clarity and understanding, these illustrations are merely illustrative and not limiting of the scope of the invention. Other embodiments, changes and modifications, including those obvious to persons skilled in the art, will be within the scope of the following claims.

What is claimed is:

1. A method of de novo designing molecules that bind to a receptor site on a protein comprising the steps of:
(a) building a molecule in the receptor site comprising: adding successive random molecular fragments to an initial molecular fragment that is loaded into the receptor site, estimating the free energy of the molecule being grown after each addition of a molecular fragment, and orienting each successive molecular fragment as it is added to the receptor site such that the free energy estimate for the molecule may be higher than a lowest free energy estimate possible for the molecule;
(b) repeating step (a) to generate a collection of molecules grown in the receptor site, and ranking the collection of molecules according to increasing free energy estimates to identify high-ranking molecules;
(c) selecting one or more functional groups of a high-ranking molecule identified in step (b) as a single restart fragment and using the restart fragment to build a second-generation of molecules according to steps (a) and (b);
(d) minimizing the energy of a protein/ligand complex comprising the receptor site and a second-generation molecule using an empirical force field
(e) quantitatively measuring the empirical interaction energy of the second-generation molecules, and ranking the molecules, wherein a molecule of low interaction strength is ranked higher than a molecule of more negative interaction energy is ranked higher than a molecule of less negative or positive interaction energy;
(f) modifying high-ranking molecules from step (e) based on qualitative analysis of the molecules including determination of chemical viability, synthetic feasibility, solubility, and effect of the molecule on the structure of the protein, whereas such modification comprises: atomic and/or functional substitutions, initiating growth from a specific receptor site, inclusion of salt bridges or hydrogen bonds, and solubility-enhancing measures;
(g) repeating steps (c) through (f) until a molecule is built which is identified as high-ranking in both steps (e) and (f).

2. The method of claim 1, wherein the receptor site is selected from the group consisting of: Src-homology-3 domain, Src-homology-2 domain, MDM2 protein, CD4 protein, and human carbonic anhydrase II protein.

3. The method of claim 1, wherein the empirical interaction energy comprises CHARMM interaction energy.

4. The method of claim 1, wherein the empirical force field comprises CHARMM.

5. A library of ligand candidates which bind to the src-2 homology domain wherein the ligand candidates are built according to a de novo structure-based design method, and comprise the following Formulas I, II and III: 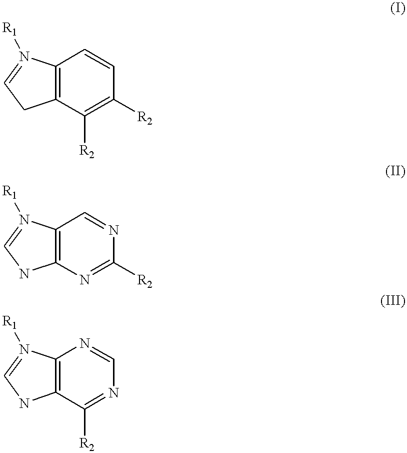
wherein
R1 is alkyl, aryl, heteroaryl, alkylaryl, arylalkyl, cycloalkenyl, cycloalkyl, cycloalkylamido, and arylalkylamido; and
R2 is independently at each occurrence selected from the group consisting of hydrogen, alkyl, aryl, heteroaryl, (heteroaryl)alkyl, alkylaryl, arylalkyl, cycloalkenyl, cycloalkyl, acyl, acyloxy, amino, amido, and alkoxy, wherein one or more groups may be optionally substituted.

6. A library of ligand candidates which bind to the MDM2 protein wherein the ligand candidates are built according to a de novo structure-based design method, and comprise the following Formula IV: 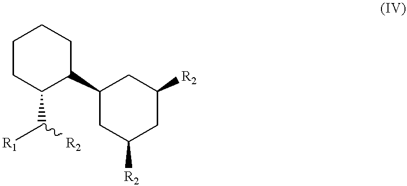
wherein
R1 is selected from the group consisting of hydrogen, alkyl, cycloalkyl, arylalkyl, aryl, heteroaryl, and (heteroaryl)alkyl, any of which groups may be optionally substituted; and
R2 is independently at each occurrence selected from the group consisting of hydrogen, alkyl, cycloalkyl, arylalkyl, aryl, heteroaryl, and (heteroaryl)alkyl, OH, O(R3), amino, wherein the aryl or heteroaryl group may be optionally substituted,
wherein R3 is selected from the group consisting of alkyl, cycloalkyl, aryl, aralkyl, (heteroaryl)alkyl, and heteroaryl, wherein the aryl or heteroaryl group may be optionally substituted.

7. A method for building molecules for binding to a receptor site present on a substrate, wherein the substrate is selected from the group consisting of Src-homology-3 domain, Src-homology-2 domain, MDM2 protein, CD4 protein, and carbonic anhydrase protein, comprising the steps of:
(a) evaluating a receptor site for a molecular make up of at least a portion of the receptor site to which a molecule being grown will bind and generating at least a coordinate of at least a portion of the receptor site to which the molecule being grown will bind, and outputting, at least with respect to the molecular make up of the receptor site, the coordinate of the portion of the receptor site to which the molecule being grown will bind;
(b) estimating free energy of the molecule being grown using knowledge-based potential data to estimate free energy and outputting the estimated free energy; and
(c) building a molecule for binding to the receptor site using the outputs from steps (a) and (b), with the building step including building the molecule by selecting molecular fragments at orientations that will result in free energy estimates for the molecule that may be higher than a lowest free energy estimate possible for the molecule.

8. A method for building molecules for binding to a receptor site present on a substrate, wherein the substrate is selected from the group consisting of Src-homology-3 domain, Src-homology-2 domain, MDM2 protein, CD4 protein, carbonic anhydrase protein, comprising the steps of:
(a) evaluating a receptor site for a molecular make up of at least a portion of the receptor site to which a molecule being grown will bind and generating at least a coordinate of at least a portion of the receptor site to which the molecule being grown will bind, and outputting, at least with respect to the molecular make up of the receptor site, the coordinate of the portion of the receptor site to which the molecule being grown will bind;
(b) estimating the free energy of the molecule being grown using knowledge-based potential data to estimate free energy and outputting the estimated free energy, comprising the substeps of:
(1) selecting an interaction radius of a length that negates adverse affects from solvent entropy effects;
(2) developing an accessible listing regarding known structures of molecule/receptor complexes that includes at least molecular fragment
(3) developing an accessible listing regarding atom types based on classes of molecular interactions that include at least atom types predicted to interact in the molecule/receptor complex of the molecule being grown and the receptor site;
(4) generating an accessible listing of molecular interactions for known structures of molecule/receptor complexes based on the selection at substep (1) and the listing developed at substeps (2) and (3);
(5) selecting a reference state for the molecule being grown that negates the adverse effects from solvent and configurational entropy effects;
(6) developing a quasichemical approximation for the molecule that is being grown; and
(7) generating the estimated free energy of the molecule being grown based on the listing developed at substep (4), the selection at substep (5), and the quasichemical approximation developed at substep (6); and
(c) building a molecule for binding to the receptor site using the outputs from steps (a) and (b), with the building step including building the molecule by selecting molecular fragments at orientations that will result in free energy estimates for the molecule that may be higher than a lowest free energy estimate possible for the molecule.

9. A method for estimating free energy of a molecule being grown for binding to a receptor site present on a substrate, wherein the substrate is selected from the group consisting of Src-homology-3 domain, Src-homology-2 domain, MDM2 protein, CD4 protein, and carbonic anhydrase protein comprising the steps of:
(a) selecting an interaction radius of a length that negates adverse affects from solvent entropy effects;
(b) developing an accessible listing of known structures of molecule/receptor complexes that includes at least atom coordinates and corresponding chemical elements;
(c) developing an accessible listing of atom types based on classes of molecular interactions that include at least atom types predicted to interact in the molecule/receptor complex of the molecule being grown and the receptor site;
(d) generating an accessible listing of molecular interactions for known structures of molecule/receptor complexes based on the selection at step (a) and the listing developed at steps (b) and (c);
(e) selecting a reference state for the molecule being grown that negates the adverse effects from solvent and configurational entropy effects;
(f) developing a quasichemical approximation for the molecule that is being grown; and
(g) generating the estimated free energy of the molecule being grown based on the listing developed at step (d), the selection at step (e), and the quasichemical approximation developed at step (f).

10. A method for building molecules for binding to a receptor site present on a substrate, wherein the substrate is selected from the group consisting of Src-homology-3 domain, Src-homology-2 domain, MDM2 protein, and CD4 protein, comprising the steps of:
(a) evaluating a receptor site for a molecular make up of at least a portion of the receptor site to which a molecule being grown will bind and generating at least a coordinate of at least a portion of the receptor site to which the molecule being grown will bind;
(b) loading at least hydrogen (“H”) atoms at least at the portion of the receptor site to which the molecule being grown will bind;
(c) randomly selecting an H atom of the H atoms loading at the receptor site;
(d) randomly selecting a molecular fragment from the predetermined group of molecular fragments and randomly selecting an H atom from the molecular fragment;
(e) forming a bond at the selected H atoms selected at steps (c) and (d);
(f) orienting the molecular fragment with respect to the bond formed at step (e) so that the molecule being grown has low free energy;
(g) estimating free energy for the molecule being grown using knowledge-based potential; and
(h) comparing the estimated free energy at step (g) with a free energy estimate of a molecule bound at the receptor site before the fragment was bonded to the receptor site at step (e) to determine if the estimated free energy at step (g) is more or less than a prior estimated free energy for the molecule before the fragment was bonded to the receptor site at step (e), and accepting the addition and orientation of the molecular fragment if the estimated free energy is less and accepting the addition and orientation of the first molecular fragment according to a probability determined in a predetermined manner if the estimated free energy is more.

11. The method as recited in claim 10, wherein orienting the molecular fragment in step (f) includes torsional rotation in fixed increments.

12. The method as recited in claim 11, where the fixed increments for torsional rotation includes fixed increments of 60°.

13. The method as recited in claim 10, wherein the method of comparing and accepting the molecule is according to a metropolis Monte Carlo selection process.

14. The method as recited in claim 10, wherein the probability in step (h) is determined according to:
$$p = exp [ - \frac{Δ g}{T} ]$$
where,
p=Probability of acceptance;
Δg=ΔG/N is the change in free energy per atom (with ΔG being the free energy difference upon adding the fragment at issue and N being the total number of atoms in the ligand);
T=Temperature.

15. The method as recited in claim 10, wherein the method further includes repeating steps (c) to (g) for each molecular fragment that is bonded.

16. The method as recited in claim 7 or 8, wherein a molecule can be grown according to steps (a) to (c) in less than 5 seconds.

17. The method of claim 7, 8, 9, or 10, wherein the molecule binds to a specificity pocket of the Src-homology-3 domain.

18. The method of claim 7, 8, 9, or 10, wherein the molecule binds to a LP pocket of the Src-homology-3 domain.

19. The method of claim 18, wherein the molecular fragment for building the molecule that binds to an LP pocket of the Src-homology-3 domain comprises proline.

20. A ligand candidate that binds to a receptor site on the CD4 protein generated de novo using a structure-based drug design method which comprises the following Formula V: [chemical structure]
